(19) United States
(12) Patent Application Publication
(10) Pub. No.: US 2026/0188434 A1
(43) Pub. Date: Jul. 2, 2026

(54) INFORMATION PROCESSING DEVICE, TRAINED MODEL GENERATION DEVICE, INFORMATION PROCESSING METHOD, TRAINED MODEL GENERATION METHOD, INFORMATION PROCESSING PROGRAM, AND TRAINED MODEL GENERATION PROGRAM

(71) Applicants: OMRON Corporation, Kyoto (JP); The University of Osaka, Osaka (JP)

(72) Inventors: Naoya CHIBA, Tokyo (JP); Yoshitaka USHIKU, Tokyo (JP); Tatsunori TANIAI, Tokyo (JP); Ryo IGARASHI, Tokyo (JP); Kanta ONO, Osaka (JP); Yuta SUZUKI, Osaka (JP); Kotaro SAITO, Osaka (JP)

(73) Assignees: OMRON Corporation, Kyoto (JP); The University of Osaka, Osaka (JP)

(21) Appl. No.: 19/128,791

(22) PCT Filed: Nov. 14, 2023
(86) PCT No.: PCT/JP2023/040970

§ 371 Date: May 9, 2025

(30) Foreign Application Priority Data
Nov. 21, 2022 (JP) 2022-186043
Aug. 9, 2023 (JP) 2023-130520

(51) Int. Cl. G16C 10/00; G06N 3/0455; G06N 3/088; G06T 17/00; G16C 20/70

(52) U.S. Cl. CPC G16C 10/00; G06N 3/0455; G06N 3/088; G06T 17/00; G16C 20/70; G06T 2210/56

(57) ABSTRACT

A field that represents the structure of a substance formed with an atomic point cloud is expressed using a neural network model.

### TECHNICAL FIELD

The present disclosure relates to an information processing device, a trained model generation device, an information processing method, a trained model generation method, an information processing program, and a trained model generation program.

### BACKGROUND ART

There are known techniques relating to autoencoder-based generative deep representation learning pipelines for 3D crystalline structures (for example, see Callum J. Court, Batuhan Yildirim, Apoorv Jain, and Jacqueline M. Cole, “3-D Inorganic Crystal Structure Generation and Property Prediction via Representation Learning”, J. Chem. inf. Model. 2020, 60, 10, 4518-4535, [searched Nov. 18, 2022], the Internet <URL: https://pubs.acs.org/doi/10.1021/acs.jcim.0c00464>).

### SUMMARY OF INVENTION

### Technical Problem

The present disclosure aims to express a field representing the structure of a substance formed with an atomic point cloud.

### Solution to Problem

To achieve the above objective, an information processing device according to the present disclosure is an information processing device including a processing unit that expresses a field representing the structure of a substance formed with an atomic point cloud, using a neural network model.

An information processing method according to the present disclosure is an information processing method implemented by a computer to perform a process of expressing a field representing the structure of a substance formed with an atomic point cloud, using a neural network model.

An information processing program according to the present disclosure is an information processing program for causing a computer to perform a process of expressing a field representing the structure of a substance formed with an atomic point cloud, using a neural network model.

### Advantageous Effects of Invention

With an information processing device, an information processing method, and an information processing program according to the present disclosure, it is possible to express a field representing the structure of a substance formed with an atomic point cloud.

### BRIEF DESCRIPTION OF DRAWINGS

FIG. 1 is a diagram for explaining the present embodiment.

FIG. 2 is a diagram for explaining the present embodiment.

FIG. 3 is a diagram for explaining the present embodiment.

FIG. 4 is a diagram for explaining the present embodiment.

FIG. 5 is a diagram for explaining the present embodiment.

FIG. 6 is a diagram for explaining the present embodiment.

FIG. 7 is a block diagram showing the hardware configuration of an information processing device according to the present embodiment.

FIG. 8 is a diagram for explaining the present embodiment.

FIG. 9 is a table for explaining the present embodiment.

FIG. 10 is a diagram for explaining the present embodiment.

FIG. 11 is a diagram for explaining the present embodiment.

FIG. 12 is a diagram for explaining the present embodiment.

FIG. 13 is a chart for explaining the present embodiment.

FIG. 14 is a chart for explaining the present embodiment.

FIG. 15 is a chart for explaining the present embodiment.

FIG. 16 is a chart for explaining the present embodiment.

FIG. 17 is a chart for explaining the present embodiment.

FIG. 18 is a chart for explaining the present embodiment.

FIG. 19 is a chart for explaining the present embodiment.

FIG. 20 is a chart for explaining the present embodiment.

FIG. 21 is a chart for explaining the present embodiment.

FIG. 22 is a chart for explaining the present embodiment.

FIG. 23 is a chart for explaining the present embodiment.

FIG. 24 is a chart for explaining the present embodiment.

FIG. 25 is a chart for explaining the present embodiment.

FIG. 26 is a chart for explaining the present embodiment.

FIG. 27 is a chart for explaining the present embodiment.

FIG. 28 is a chart for explaining the present embodiment.

FIG. 29 is a chart for explaining the present embodiment.

FIG. 30 is a chart for explaining the present embodiment.

### DESCRIPTION OF EMBODIMENTS

In the following, an example of an embodiment of the present disclosure is described, with reference to the drawings. In the present embodiment, an information processing device according to the present disclosure is described as an example. Note that, in the respective drawings, the same or equivalent components and portions are denoted by the same reference numerals. Further, the dimensions and ratios in the drawings are exaggerated for convenience sake, and might be different from actual ratios.

### Overview

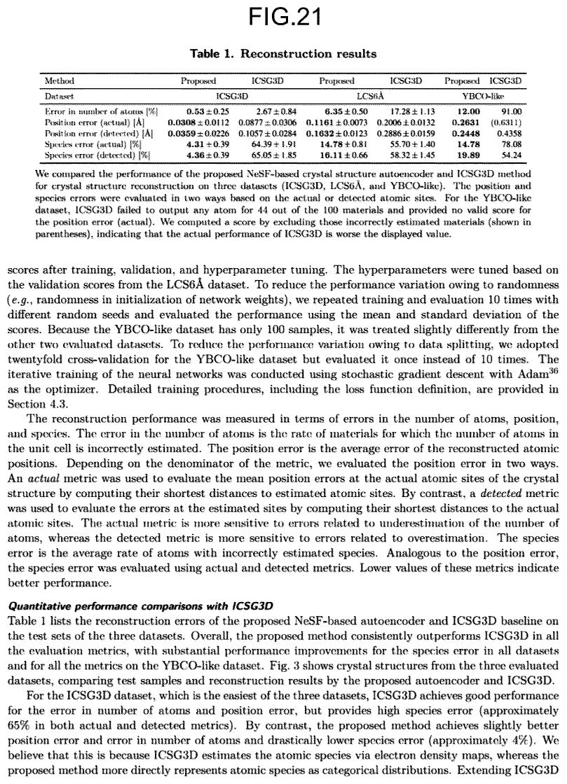

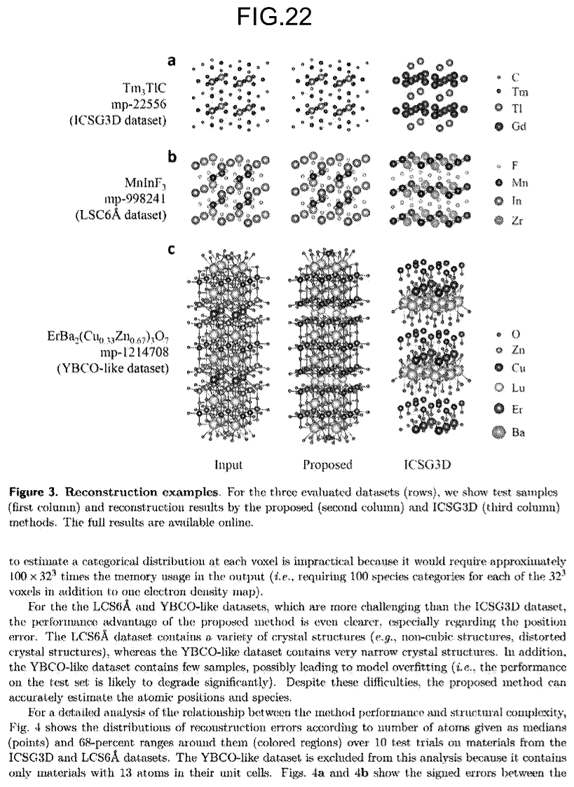

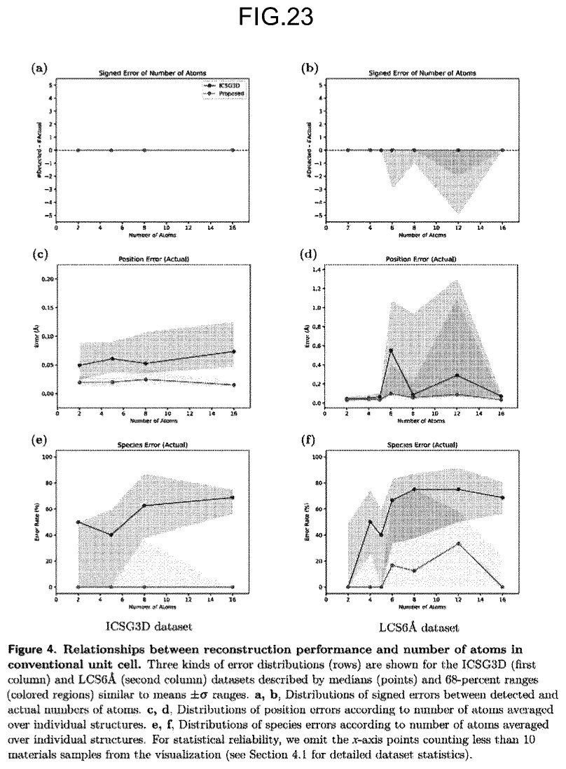

A method of inverse design of crystals, particularly materials, can contribute to next generation methods of searching for materials having the desired properties, without relying on “luck” or “serendipity”. In the present embodiment, neural structure fields (NeSF) is proposed as an accurate and practical approach for representing crystal structures using neural networks. Inspired by the concepts of vector fields in physics and implicit neural representations in computer vision, the proposed NeSF considers a crystal structure as a continuous field, rather than a discrete set of atoms. Unlike existing grid-based discretized spatial representations, the NeSF overcomes the tradeoff between spatial resolution and computational complexity, and can represent any crystal structure. To evaluate the NeSF, the present embodiment proposes an autoencoder of crystal structures that can recover various crystal structures, such as those of perovskite structure materials and cuprate superconductors. Extensive quantitative results demonstrate the superior performance of the NeSF compared with the existing grid-based approach. Note that, in the following description, the numbers in brackets “[ ]” indicate the numbers assigned to the references shown in FIGS. 21 to 23.

### 1. Introduction

A fundamental paradigm of materials science considers structure-property relationships assuming that the material properties are tightly coupled with their crystal structures. Therefore, for traditional approaches in materials science, theoretical and experimental analyses of the structure-property relationships of materials are conducted in search for novel materials having superior properties [1, 2]. However, these traditional approaches rely on labor-intensive human analysis and even “serendipity”. To automate or assist the material analysis and development, data-driven approaches have been actively studied in materials science and the area of materials informatics (MI) has been established [3-6].

Unlike traditional approaches based on the deduction of physical laws, MI aims to unveil materials knowledge (such as laws governing structure-property relationships) from datasets of collected materials via statistical and machine learning (ML) methods. In recent years, MI has been developed rapidly owing to technological advances in ML and the advent of large-scale materials databases [6, 7]. Thus, powerful neural-network-based ML methods are becoming key components in MI research [6]. Applications of MI include the prediction of material properties from material characteristic data such as crystal structures [6] and compositions [8-11], automated analyses of experimental data [12-14], and natural language processing for knowledge retrieval from scientific literature [15].

Many MI studies have been focused on predicting the properties of given materials, such as the bandgap, Seebeck coefficient, and elastic modulus [16-18]. When this type of task is regarded as the discovery of structure-to-property relationships among materials, there is yet another important type of task, which is the discovery of property-to-structure relationships that constitutes an inverse design problem [19-23].

Despite the potential utility of this inverse approach in developing materials, few studies have addressed this [19-23] or its underlying problem [24-26], that is, the estimation of crystal structures under given conditions. Regarding MI and ML, whether to input or output crystal structures (that is, whether to encode or decode crystal structures in MI and ML terms) induces a crucial difference. Although encoding crystal structures is suitably established using graph neural networks [10, 16, 17, 27], a technical bottleneck remains in decoding crystal structures. In this study, the bottleneck was addressed.

The crystal structure of an inorganic material is a regular and periodic arrangement of atoms in a three-dimensional (3D) space. This arrangement is usually described by the 3D positions and species of atoms in a unit cell and the lattice constants defining the movement of the unit cell in 3D space. The atoms in a unit cell have no explicit order, and their quantity varies from one to hundreds in number. Because ML models, including neural networks, generally accept fixed-dimensional and consistently ordered tensors for processing, handling crystal structures with ML models is not easy [16], and determining crystal structures via the models is even more difficult.

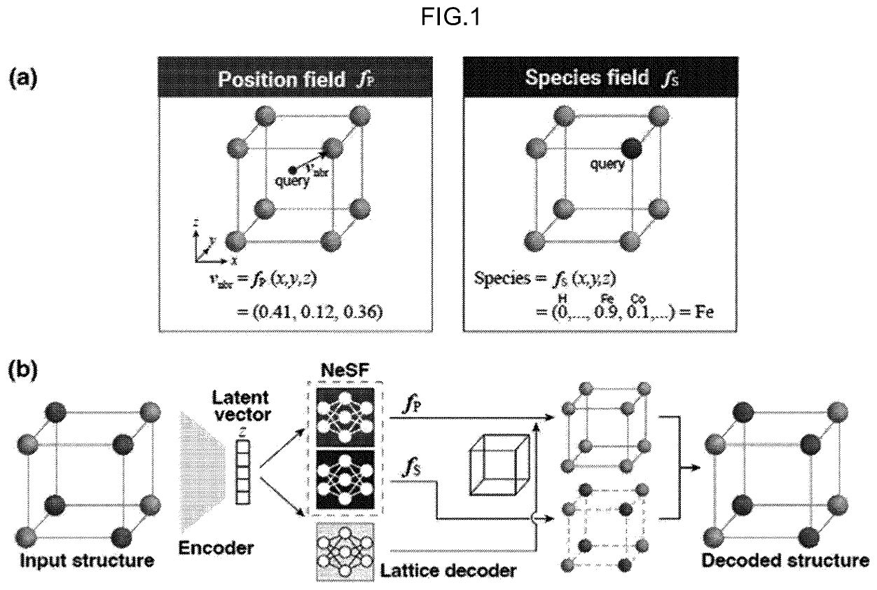

The present embodiment proposes a general representation of crystal structures that enables neural networks to decode or determine such structures. The key concept underlying our approach is illustrated in FIG. 1. Here, a crystal structure is not represented as a discrete set of atoms, but as a continuous vector field tied to 3D space. We refer to our approach as neural structure fields (NeSF). The NeSF uses two types of vector fields, which are the position and species fields, to implicitly represent the positions and species of the atoms in the unit cell of the crystal structure, respectively.

To illustrate the concept of the NeSF, it is assumed that target material information is given as a fixed-dimensional vector z, and the problem of recovering a crystal structure from z is considered. The input z can specify, for example, information about the crystal structure of a material or some desired criteria for materials to be produced. In the NeSF, a neural network f is not made to directly output the crystal structure as f(z), but the neural network f is used as an implicit function to indirectly represent the crystal structure embedded in z. Specifically, f is treated as a vector field on 3D Cartesian coordinates p, conditioned on the target material information z.

$$\begin{matrix}[ Math . 1 ] & \\ s = f ( p , z ) & ( 1 )\end{matrix}$$

In the position field, the network f is trained to output a 3D vector pointing from a query point p to its nearest atomic position a in the crystal structure of interest. Thus, an output s is expected to be a−p. In a case where the position field is ideally trained, the position a of the nearest atom at any query point p can be obtained as p+f(p, z).

Mathematically, the position field can be interpreted as the gradient vector field −φ(p) of the scalar potential shown below.

$$\begin{matrix}[ Math . 2 ] & \\ ϕ ( p ) = \frac{1}{2} {min}_{i} {\vert p - {a}_{i} \vert}^{2} & \end{matrix}$$

The scalar potential is represented by the squared distance between the query point p and the position {aᵢ} of the nearest atom. Likewise, the species field is trained to output a categorical probability distribution that indicates the species of the nearest atom. Accordingly, the output dimension of the species field is the number of candidate atomic species.

The proposed NeSF is inspired by the concept of vector fields in classical physics and implicit neural representations [30-34] in computer vision. Implicit neural representations have recently been proposed to handle some representation issues in 3D computer vision applications, such as 3D shape estimation of objects [31-33] and free-viewpoint image synthesis [30, 34]. In 3D shape estimation, when a neural network is made to directly output a 3D mesh or point cloud, representation issues similar to those occurring in crystal structures occur. To overcome these issues, the signed distance function (SDF) is utilized in DeepSDF [31] to model 3D shapes by causing the neural network f(p) to indicate whether the query point p is outside or inside the object volume with a positive or negative sign in the respective scalar outputs. The NeSF follows the basic idea of implicit neural representations and further extends it to the estimation of crystal structures described by atomic positions and species. The precise description of atomic positions in crystal structures is of crucial interest in materials science. Thus, the NeSF outputs vectors pointing to the nearest atoms to represent atomic positions more directly than existing implicit neural representations of 3D geometries.

Our idea of representing 3D geometry crystal structures as continuous vector fields has been partially and implicitly explored using grid-based discretization (which is voxelization) in recent MI studies [19, 20, 22, 24-26], but without explicit consideration as discretized vector fields. In those studies, the 3D space in the unit cell is discretized into voxels, and each voxel is then assigned an electron density. The electron density basically represents the presence or absence of an atom around the voxel. However, compared with one-dimensional (such as audio signals) or two-dimensional (such as images) data, the discretization of 3D data suffers considerably from the tradeoff between spatial resolution and computational complexity, in terms of both computational time and memory space. For example, the ICSG3D method [24] uses 32×32×32 voxels to represent the crystal structures and estimates them using 3D convolutional neural networks (CNNs). Because voxel-based 3D CNNs involve a lot of computation and memory, the resolution of 32×32×32 voxels is an approximate limit for training a voxel-based model on a standard computing system. Meanwhile, existing crystal structures contain tens or more atoms in their unit cells, or have elongated or distorted unit cells. Therefore, accurately representing diverse crystal structures with voxels requires a sufficiently high resolution. Moreover, voxel-based models can only indirectly provide atomic positions in representations such as peaks in a scalar field of electron densities discretized in the voxel space. The proposed NeSF overcomes the limitations of voxelization. In the NeSF, there is essentially no tradeoff between spatial resolution and required memory. Theoretically, the NeSF can achieve infinitely high spatial resolution with compact (memory- and parameter-efficient) neural networks in place of costly 3D CNNs. In addition, the NeSF can effectively represent any crystal structures including those with elongated or distorted unit cells. Furthermore, the NeSF can directly provide the Cartesian coordinates of the atomic positions rather than peaks of a scalar field. It is believed that the proposed NeSF will break through the technical bottleneck of the MI approach for crystal structure estimation and contribute to the advancement of MI research in this direction.

In the following section, the proposed NeSF is described in detail, and its expressive power for various crystal structures is demonstrated through numerical experiments. Notably, the NeSF successfully recovers various crystal structures, from the relatively basic structures of perovskite materials to the complex structures of cuprate superconductors. Results from extensive quantitative evaluations show that the NeSF outperforms the voxelization approach in ICSG3D [24].

### 2 Results and Discussion

First, the procedures for estimating crystal structures with the NeSF and training the NeSF are described. An autoencoder of crystal structures is then presented as an application of the NeSF. In this autoencoder, crystal structures are embedded into vectors z (called latent vectors) via an encoder, and the NeSF then acts as a decoder to recover the input crystal structures from z. The performance of the NeSF-based autoencoder is quantitatively analyzed by evaluating the reconstruction accuracy, outperforming the voxelization-based ICSG3D baseline. The space of learned vectors z is qualitatively analyzed. With the proposed autoencoder, this analysis shows that the learned space reflects some similarity between crystal structures, instead of merely embedding crystal structures at random.

### <2.1 Crystal Structures by NeSF>

When estimation target material information is given as a vector z, estimating the crystal structure from z is estimating the positions and species of the atoms in the unit cell along with the lattice constants. The lattice constants are modeled as lengths a, b, and c and angles α, β, and γ, and are estimated via simple multilayer perceptrons (MLPs) with the input z. On the other hand, the atomic positions and species are estimated by the position field fₚ and the species field fₛ of the NeSF, respectively, as described in the previous section. These fields are also implemented as simple MLPs, each taking the query position p and the vector z as inputs and predicting a field value (which is a 3D pointing vector or a categorical probability distribution). An overview of the NeSF network architecture is illustrated in the right part of FIG. 1(b).

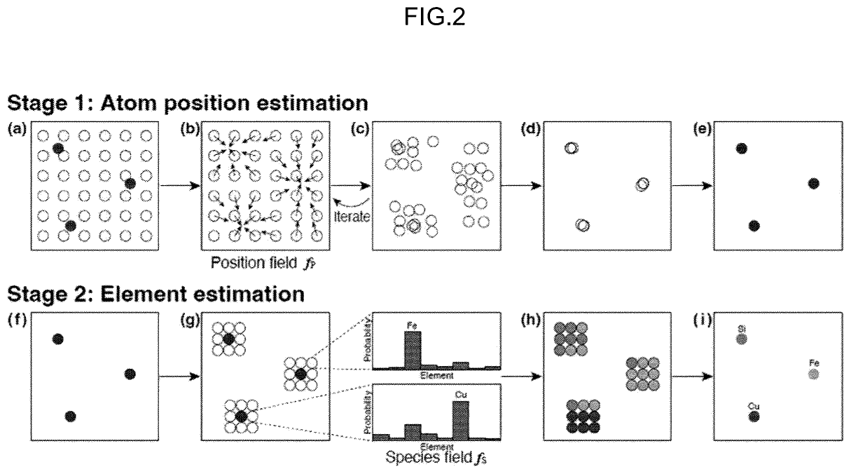

Given the vector z from the encoder, the estimation of the atomic positions and species using the NeSF is illustrated in FIG. 2 and is summarized in five steps.
1. Initialize particles. First, the lattice constants are estimated via MLPs. Initial query points {p⁰ᵢ} called particles are then regularly spread at 3D grid points within a bounding box. The bounding box is common to each dataset and is given to loosely encompass the atoms of all training samples.
2. Move particles. The position of each particle p^{t}ᵢ is updated using the position field according to p^{t+1}ᵢ=p^{t}ᵢ+fₚ(p^{t}ᵢ, z). This process is repeated for all particles, to obtain {pᵢ} as candidate atomic positions. Because the position field is expected to point to the nearest atomic position, the particles travel toward their nearest atoms through this process.
3. Score particles. Each particle pᵢ is scored, and outliers are filtered. As he norm ∥fₚ(pᵢ, z)∥ of the output of the position field indicates an estimated distance from pᵢ to its nearest atom, each particle pᵢ is scored by ∥fₚ(pᵢ, z)∥ and, if the score exceeds a certain threshold (set to 0.9 Å in this case), the particle is discarded.
4. Detect atoms. Until this point of time, the particles are expected to form clusters around atoms. Therefore, a simple clustering algorithm is adopted to detect each cluster position as an atomic position, and the number of atoms in the crystal structure is determined. Here, a clustering algorithm well known in object detection and non-maximum suppression is used. Specifically, a list of candidate particles is initialized as B_{c}={pᵢ}, and another list of accepted particles is initialized as Bₐ={ }. 1) The particle with the lowest score (estimated to be nearest to an atom) is selected from B_{c}, and is moved to Bₐ. 2) Particles within a spherical area around the selected particle are removed from B_{c} (the spherical radius was set to 0.5 Å in this study). These steps are repeated until Be becomes empty, so that the atomic position {aᵢ} as the selected particle stored in Bₐ is obtained.
5. Estimate species. Finally, the species field fₛ(p, z) is used to estimate the atomic species at each atomic position aᵢ. For robust estimation against errors in aᵢ, new particles are intensively spread as queries to the species field around each aᵢ, instead of using aᵢ directly as a query. Thus, a plurality of probability distributions is obtained, each of the probability distributions predicts the species of an atom aᵢ. The most frequent atomic species among them is selected as the final estimate.

### <2.2 Training of NeSF>

The training of the NeSF differs from the above estimation algorithm and is much simpler. Sampling is randomly performed on 3D query points {p^{s}ᵢ} in the unit cell, and loss values for the field outputs at these points are computed. Thus, fₚ(p^{s}ᵢ, z) and fₛ(p^{s}ᵢ, z) are supervised to indicate the position and species of the nearest atom, respectively. However, query points cannot be sampled densely because of practical limitations in memory usage. Therefore, training requires a sampling strategy for query points. Existing implicit neural representations for 3D shape estimation, such as DeepSDF [31], sample the training query points near the surface. Curriculum DeepSDF [35] further introduces curriculum learning, in which the sampling density intensifies near the surface as training proceeds.

To consider a desired sampling strategy for training the position field and the species field, their dynamics are considered in the proposed algorithm. 1) Particles repeatedly move in the position field toward their nearest atoms. Therefore, the position field should be sufficiently accurate anywhere to allow the flow of particles to their destinations, and be highly accurate in the vicinity of atoms. 2) The species field is queried only around atoms. Therefore, the species filed does not need to be accurate everywhere, but should be robust to errors in the estimated atomic positions.

To meet the above requirements, two sampling methods are introduced to train the position field and the species field. 1) Global grid sampling: This method considers 3D grid points that uniformly cover the entire unit cell, and samples the points with perturbations that follow a Gaussian distribution. 2) Local grid sampling: This method considers local 3D grid points centered at each atomic position, and samples the points with perturbations that follow a Gaussian distribution.

To train the position field, the two sampling methods are combined. Accordingly, query points are sampled uniformly over the unit cells and densely around the atoms. To train the species field, local grid sampling is used to concentrate training query points in the vicinity of atoms.

### <2.3 Crystal Structure Autoencoder>

To demonstrate and evaluate the expressive power of the NeSF, an autoencoder of crystal structures is proposed. Like other common autoencoders, the proposed NeSF-based autoencoder includes an encoder and a decoder. The encoder is a neural network that transforms an input crystal structure (which is the positions and species of the atoms in the unit cell, and lattice constants) into an abstract latent vector z. The decoder, for which the NeSF is used, reconstructs the input crystal structure from the latent vector z. Autoencoders are typically used to learn latent vector representations of data via self-supervised learning. In this learning, the input data can supervise the learning via a reconstruction loss.

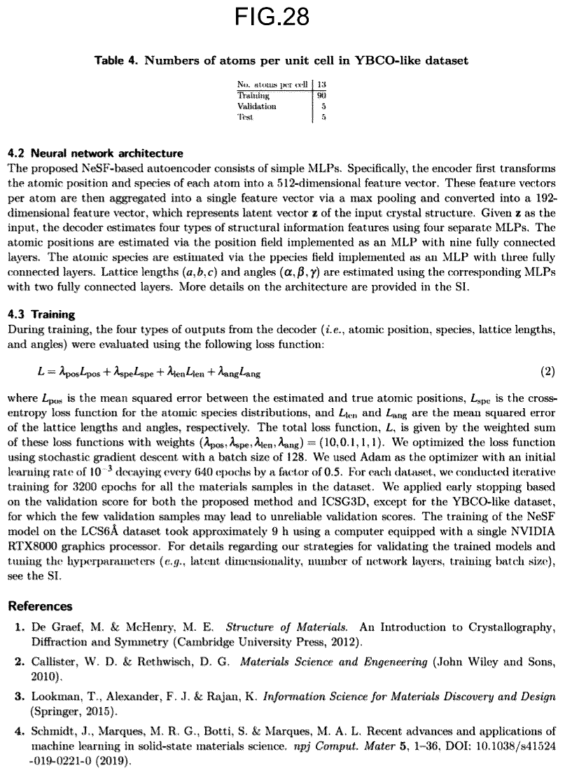

While we focus on decoding crystal structures, their encoding is studied in MI. Because a crystal structure is essentially a set of atoms, its encoding needs to handle a variable number of atoms with invariance to permutation. In ML, such encoders are generally called set functions [28, 29]. Among them, the family of graph neural networks [10, 16, 17, 27] serves as popular crystal structure encoders. However, these networks implicitly represent atomic positions as edges, and encode distances between atoms while discarding the exact coordinates. While this distance-based graph representation is a key to ensuring invariance to coordinate system, its information loss in input might unintentionally hinder the reconstruction performance. Therefore, the basic encoder architecture from PointNet [28] and DeepSets [29] is adopted. This architecture not only represents the simplest type of set-function-based networks but can also preserve information about input crystal structures, and thus, is suitable for evaluation of performance of the NeSF decoders. The architecture of the proposed autoencoder is detailed in Section 4.2 in FIG. 28.

### <Training and Evaluation Procedures>

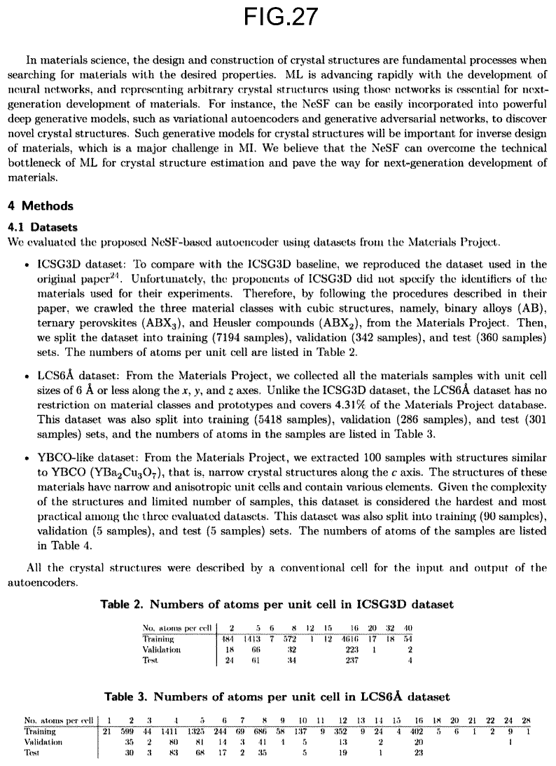

The autoencoder and ICSG3D24 (baseline) were trained and evaluated on three material datasets: ICSG3D, limited cell size 6 Å (LCS6 Å), and YBCO-like datasets. These datasets are designed to collect the crystal structures of materials from the Materials Project, and have different difficulty levels. The ICSG3D dataset [24] is a materials collection, and includes three datasets containing 7897 materials with limited crystal systems (cubic) and prototypes (which are AB, ABX2, and ABX3). The LCS6 Å dataset is formed with 6005 materials having a unit cell size of 6 Å or smaller along the x-, y-, and z-axes, and there are no restrictions on the crystal systems and prototypes. The YBCO-like dataset is formed with 100 materials having narrow unit cells along the c-axis. These structures typically include structures of yttrium barium copper oxide (YBCO) superconductors. Due to the complexity of the structure and relatively few samples, the YBCO-like dataset is the most challenging among the three evaluated datasets. Further details on these datasets are provided in Section 4.1 in FIG. 27.

For training and evaluation, each dataset was randomly split into training (90.25%), validation (4.75%), and test (5%) sets. The training set was only used to train the ML models. The validation set was used to preliminarily validate the trained ML model, and the test set was used to compute the final evaluation scores after training, validation, and hyperparameter tuning. The hyperparameters were tuned based on the validation scores from the LCS6 Å dataset. To reduce the performance variation due to validation score randomness (such as randomness in initialization of network weights), training and evaluation were repeated 10 times with various random seeds, and the performance was evaluated using the mean and standard deviation of the scores. Because the YBCO-like dataset has only 100 samples, the YBCO-like dataset was processed in a slightly different manner from the other two evaluated datasets. To reduce the performance variation due to data splitting, twentyfold cross-validation was adopted for the YBCO-like dataset, but the YBCO-like dataset was evaluated once, not 10 times. The iterative training of the neural networks was conducted using stochastic gradient descent with Adam [36] as the optimizer. Detailed training procedures, including the loss function definition, are provided in Section 4.3 in FIG. 28.

The reconstruction performance was measured in terms of errors in the number, positions, and species of atoms. The error in the number of atoms is the ratio of materials for which the number of atoms in the unit cell is incorrectly estimated. The position error is the mean error of the reconstructed atomic positions. Depending on the denominator of the metric, the position error was evaluated by two methods. An actual metric was used to evaluate the mean position errors at the actual atomic sites of the crystal structure by computing their shortest distances to estimated atomic sites. In contrast, a detected metric was used to evaluate the errors at the estimated sites by computing their shortest distances to the actual atomic sites. The actual metric is more sensitive to errors related to underestimation of the number of atoms, while the detected metric is more sensitive to errors related to overestimation. The species error is the average rate of atoms whose species are incorrectly estimated. Like the position error, the species error was evaluated using actual and detected metrics. Lower values of these metrics indicate better performance.

<Quantitative Performance Comparisons with ICSG3D>

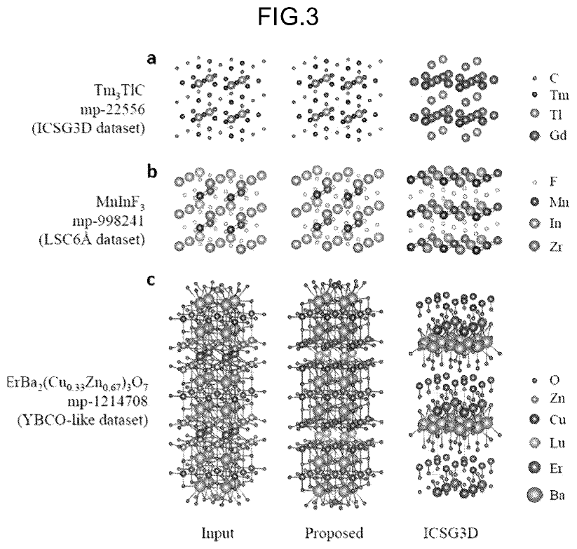

Table 1 shows the reconstruction errors of the proposed NeSF-based autoencoder and ICSG3D baseline on the test sets of the three datasets. Overall, the proposed method consistently outperforms ICSG3D in all the evaluation metrics, with substantial performance improvements for the species error in all datasets and for all the metrics on the YBCO-like dataset. FIG. 3 shows crystal structures from the three evaluated datasets, comparing test samples and reconstruction results by the proposed autoencoder and ICSG3D.

TABLE 1
Reconstruction results

| | Method | | | | | |
|---|---|---|---|---|---|---|
| | Proposed | ICSG3D | Proposed | ICSG3D | Proposed | ICSG3D |
| | Dataset | | | | | |
| | ICSG3D | | LCS6Å | | YBCO-like | |
| [symbol] [ [symbol] ] | [symbol] ± [symbol] | [symbol] ± [symbol] | [symbol] ± [symbol] | [symbol] ± [symbol] | [symbol] | [symbol] |
| [symbol] [Å] | [symbol] ± [symbol] | [symbol] ± [symbol] | [symbol] ± [symbol] | [symbol] ± [symbol] | [symbol] | ( [symbol] ) |
| [symbol] [Å] | [symbol] ± [symbol] | [symbol] ± [symbol] | [symbol] ± [symbol] | [symbol] ± [symbol] | [symbol] | [symbol] |
| [symbol] [ [symbol] ] | [symbol] ± [symbol] | [symbol] ± [symbol] | [symbol] ± [symbol] | [symbol] ± [symbol] | [symbol] | [symbol] |
| [symbol] [ [symbol] ] | [symbol] ± [symbol] | [symbol] ± [symbol] | [symbol] ± [symbol] | [symbol] ± [symbol] | [symbol] | [symbol] |

We compared the performance of the proposed NeSF-based crystal structure autoencoder and ICSG3D method for crystal structure reconstruction on three datasets (ISCG3D, LCS6Å, and YBCO-like). The position and species errors were evaluated in two ways based on the actual or detected atomic sites. For the YBCO-like dataset, ICSG3D failed to output any atom for 44 out of the 100 materials and provided no valid score for the position error (actual). We computed a score by excluding those incorrectly estimated materials (shown in parentheses), indicating that the actual performance of ICSG3D is worse the displayed value. [symbol]
indicates data missing or illegible when filed

In the case of the ICSG3D dataset, which is the simplest of the three datasets, ICSG3D achieved good performance for the error in number of atoms and the position error, but provides a large species error (about 65% in both actual and detected metrics). In contrast, the proposed method achieves slightly smaller errors in position and in number of atoms, and a dramatically smaller species error (about 4%). This is likely because ICSG3D estimates atomic species via electron density maps, while the proposed method more directly represents atomic species as categorical distributions. Extending ICSG3D to estimate a categorical distribution at each voxel is impractical, because it would require approximately 100×323 times the memory usage in the output (that is, 100 species categories are required for every 323 voxels in addition to one electron density map).

In the cases of the LCS6 Å and YBCO-like datasets, which are more challenging than the ICSG3D dataset, the performance advantage of the proposed method is even clearer, especially with regard to the position error. The LCS6 Å dataset contains a variety of crystal structures (such as non-cubic structures and distorted crystal structures), while the YBCO-like dataset contains very narrow crystal structures. Further, the YBCO-like dataset contains few samples, which might lead to model overfitting (which means that the performance on the test set is likely to deteriorate significantly). Despite these difficulties, the proposed method can accurately estimate the atomic positions and species.

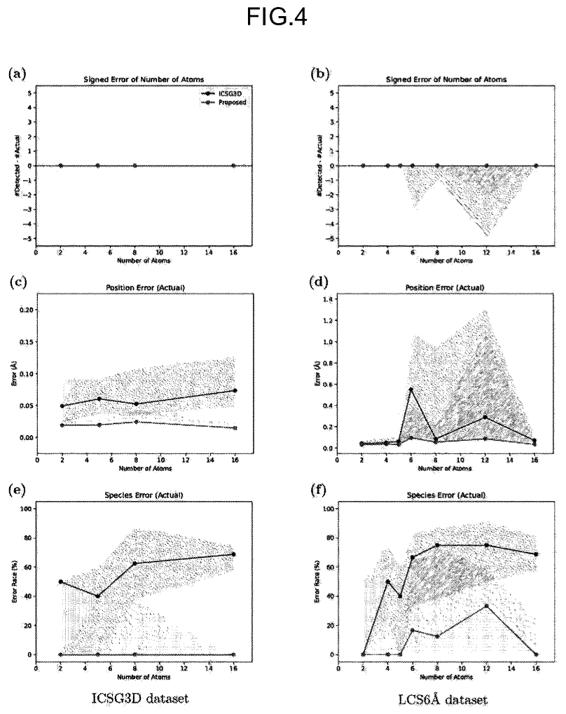

For a detailed analysis of the relationship between method performance and structural complexity, FIG. 4 shows the distributions of reconstruction errors depending on the number of atoms given as medians (points) and the 68% range (colored regions) around them over ten test trials on materials from the ICSG3D and LCS6 Å datasets. The YBCO-like dataset is excluded from this analysis, because the YBCO-like dataset contains only materials with 13 atoms in their unit cells. FIGS. 4a and 4b show the signed errors between the number of detected atoms and the number of actual atoms. These results indicate that both methods correctly estimate the number of atoms for most (68% or more) of the samples in the ICSG3D dataset (FIG. 4a). However, the ICSG3D method underestimates the number of atoms for the LCS6 Å dataset (FIG. 4b). Likewise, FIGS. 4c and 4d show the distributions of position errors, and FIGS. 4c and 4d show the distributions of position error. FIGS. 4e and 4f show the distributions of species errors. Because the number of atoms is either correctly estimated or underestimated by both methods in most cases, errors are reported in the actual metrics. By examining the distributions at x=2 in FIG. 4e, it is possible to determine the tendency of species errors for the diatomic structures in the ICSG3D dataset.

While the proposed method provides the correct species of both atoms for 68% or more of the diatomic materials, ICSG3D often misestimates the species of one of the two atoms. Overall, the three types of errors by both methods tend to increase with the number of atoms, but the proposed method consistently achieves better results than ICSG3D for materials with varying numbers of atoms. Compared with our method, the performance of ICSG3D tends to deteriorate more notably for materials with large numbers of atoms. ICSG3D tends to underestimate the number of atoms (FIG. 4b), which is likely due to the spatial resolution of ICSG3D limited to 32×32×32 voxels. This suggests that ICSG3D is incapable of capturing polyatomic structures.

It is believed that the notably high performance of the proposed method is attributable to two reasons. First, because our method does not use discretization, it is advantageous over the grid-based ICSG3D for estimating complex crystal structures. In a grid-based method, the spatial resolution is limited by the cubically increasing computations and memory usage. The proposed NeSF is free from such tradeoff between resolution and computational complexity, and thus, is able to effectively represent complex structures. Second, the model size of the proposed NeSF using MLPs is much smaller than the model size of the 3D CNN architecture of ICSG3D. In general, the number of training samples required for an ML model correlates with the number of trainable parameters. Grid-based methods use layers of 3D convolution filters that involve many trainable parameters. In contrast, the NeSF uses implicit neural representations to indirectly describe the 3D space as a field rather than voxels. Thus, the NeSF is efficiently implemented by MLPs with fewer parameters than a 3D CNN. Specifically, the NeSF-based autoencoder has 760,000 parameters, which is only 2.24% of the number of parameters in the 3D CNN-based ICSG3D (34 million parameters). This difference makes the NeSF advantageous over grid-based methods, especially on small datasets such as the YBCO-like dataset.

### <2.4 Latent Space Interpolation>

The characteristics of the proposed NeSF-based autoencoder were qualitatively analyzed by inspecting the learned latent space of the crystal structures. In general, a good latent space should map similar items (in terms of properties, characteristics, categories, and the like) closely in the space. In this manner, latent data representations that facilitate human and machine analyses are provided. To assess the construction of the latent space of crystal structures, transitions in the latent space were visualized as sequences of crystal structures. In a case where the latent space is trained to capture relationships between materials in terms of structural similarity, interpolating between two points in the latent space should produce a sequence of materials with similar crystal structures.

Interpolation analysis proceeds as follows.
1. Set the ICSG3D test set as the source and destination materials, and obtain their latent vectors as z_{src} and z_{dst} via the trained encoder.
2. Interpolate between z_{src} and z_{dst} linearly in the latent space to obtain a sequence of latent vectors.
3. Decode each latent vector via the trained decoder (NeSF) to obtain its crystal structure.

Because the intermediate crystal structures between the source and the destination is reconstructed from the latent vector via the trained decoder, these structures might not appear in the dataset.

To facilitate interpretation of the analysis, the well-known zinc-blende and rock-salt structure families were selected as benchmark materials. Both families have compositions given by AX, where A is a cation and X is an anion, and the crystal structure is based on a cubic crystal system. Therefore, in a case where the source and destination materials belong to one of these families, the characteristic composition and structural prototype need to be preserved throughout the interpolation path.

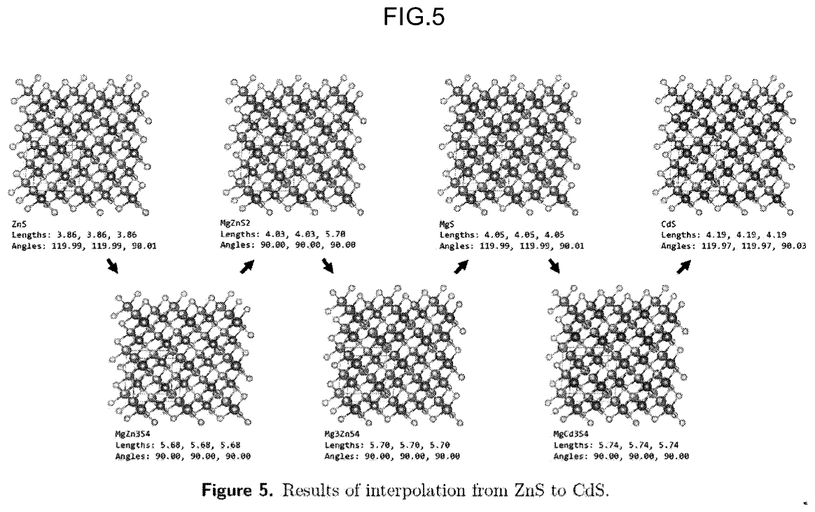

As a first example, FIG. 5 shows the results of interpolation from ZnS (mp-10695) to CdS (mp-2469). The obtained transition in the compositional formula is ZnS→MgZn₃S₄ (Mg_{0.25}Zn_{0.75}S)→MgZnS₂ (Mg_{0.5}Zn_{0.5}S)→Mg₃ZnS₄ (Mg_{0.75}Zn_{0.25}S)→MgS→MgCd₃S₄ (Mg_{0.25}Cd_{0.75}S)→CdS.

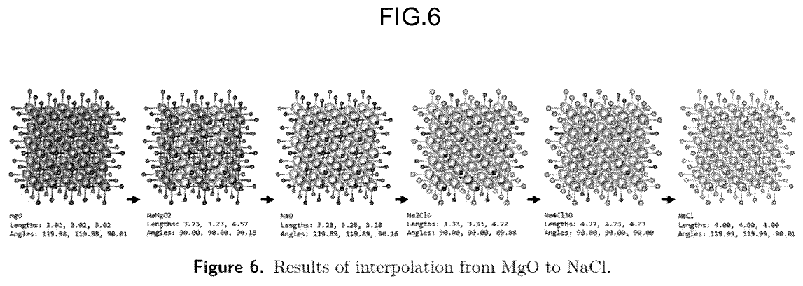

As a second example, FIG. 6 shows the results of interpolation from MgO (mp-1265) to NaCl (mp-22862). The obtained transition in the compositional formula is MgO→NaMgO₂ (Na_{0.5}Mg_{0.5}O)→NaO→Na₂ClO (NaCl_{0.5}O_{0.5})→Na₄Cl₃O (NaCl_{0.75}O_{0.25})→NaCl.

Further, in the SI, the results of interpolation from NaCl (mp-22862) to PbS, from MgO (mp-1265) to CaO (mp-2605), and from PbS (mp-21276) to CaO (mp-2605) are provided.

In the examples of interpolation, the composition AX and the cubic structure are mostly preserved. Furthermore, the compositions change continuously without collapsing along the interpolation paths. These results suggest that our encoder learns meaningful continuous representations of crystal structures by capturing their characteristics in an abstract space, and the proposed NeSF model successfully decodes these representations into crystal structures.

### <2.5 Limitations and Future Directions>

This study was mainly focused on developing a fundamental approach for crystal structure estimation using implicit neural representations. To suggest room for further improvement and important directions in future work, three main limitations of the NeSF were identified.

First, the NeSF adopts a relatively simple network architecture, and architectural design choices were not thoroughly explored. For example, the NeSF treats the elements as mutually independent categorical (one-hot) vectors. Thus, there is no explicit training of the model using the physical characteristics of elements or their similarities. Meanwhile, other developments have attempted to explicitly inject physical characteristics and functions of elements into their models. For example, existing crystal-structure encoders [16] represent input elements using their fingerprints, such as their groups and periods, electron densities, atomic radii, and electronegativities, instead of using one-hot vectors. In ICSG3D24, the outputs of atomic species are trained using the mean square error of the atomic numbers rather than a categorical loss, which is considered in our method. Incorporating these schemes into the model might be able to reflect the characteristics and similarities between elements in the latent space and reconstructed crystal structures. Another architectural choice regarding the NeSF is whether to use the existing (current choice) or primitive cell to represent crystal structures. Both formats have their advantages and drawbacks depending on the application. While the existing unit cell provides more intuitive visualizations that facilitate manual analysis, the primitive unit cell provides more compact structure representations that might facilitate computer processing. In future work, the network architecture design needs to be thoroughly analyzed.

Another limitation is that the proposed NeSF does not explicitly consider space-group symmetry. Therefore, the local spatial arrangements of atoms in the existing unit cells estimated by the NeSF do not necessarily obey the space-group symmetry. Although ICSG3D [24] shares the same limitation, symmetry is an important concept in crystallography. Therefore, incorporating the constraint of space-group symmetry into the NeSF is an important direction of future work.

Finally, for an evaluation purpose, the autoencoder architecture was adopted, instead of generative models such as variational autoencoders [16, 25, 37] and generative adversarial networks [26, 37-39]. These generative models intentionally perturb latent structural representations to produce diverse structures that do not appear in the dataset. While this aspect of the generative models is more suitable for novel structure discovery, the lack of ground-truth structures prevents quantitative and reliable performance analysis.

The NeSF needs to be applied to generative models in future work using appropriate performance analysis.

### 3 Conclusion

In the present embodiment, the NeSF to estimate crystal structures using neural networks is proposed. It is difficult to directly determine crystal structures using neural networks. This is because these structures are essentially represented as an unordered set including varying numbers of atoms. The NeSF overcomes this difficulty by treating the crystal structure not as a discrete set of atoms, but as a continuous vector field. The idea of the NeSF is borrowed from vector fields in physics and the recent implicit neural representations in computer vision. An implicit neural representation is an ML technique to represent 3D geometries using neural networks. The NeSF extends this technique by introducing the position field and the species field to estimate the atomic positions and the species of crystal structures, respectively. Unlike existing grid-based approaches for representing crystal structures, the NeSF is free from the tradeoff between spatial resolution and computational complexity, and can represent any crystal structure.

The NeSF was applied as an autoencoder for crystal structures, and demonstrated its performance and expressive power on datasets having diverse crystal structures. A quantitative performance analysis showed a clear advantage of the NeSF-based autoencoder over an existing grid-based method, especially for estimating complex crystal structures. Furthermore, a qualitative analysis of the learned latent space revealed that the autoencoder does not randomly map crystal structures, but rather captures similarities between crystal structures.

In materials science, the design and construction of crystal structures are fundamental processes in searching for materials having the desired properties. ML is advancing rapidly with the development of neural networks, and representing any crystal structures using those networks is essential for next-generation development of materials. For example, the NeSF can be easily incorporated into powerful deep generative models such as variational autoencoders and generative adversarial networks, and discover new crystal structures. Such generative models of crystal structures will be important for inverse design of materials, which is a major challenge in MI. The NeSF can overcome the technical bottleneck of ML in crystal structure estimation, and pave the way to next-generation development of materials.

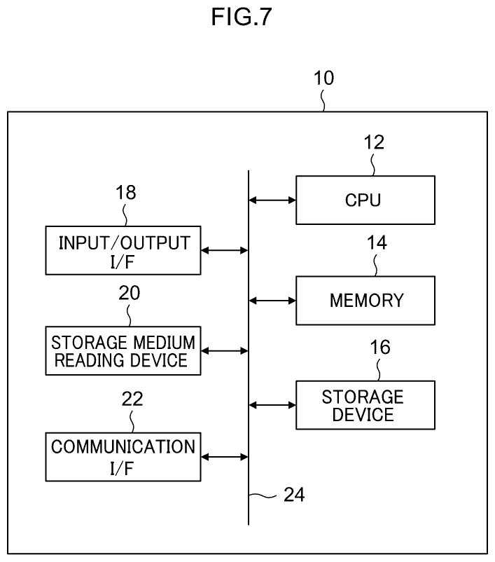

FIG. 7 is a block diagram showing the hardware configuration of an information processing device 10 according to the present embodiment. As illustrated in FIG. 7, the information processing device 10 includes a central processing unit (CPU) 12, a memory 14, a storage device 16, an input/output interface (I/F) 18, a storage medium reading device 20, and a communication I/F 22. The respective components are communicably connected to one another via a bus 24.

The storage device 16 stores an information processing program for executing each of the processes described later. The CPU 12 is a central computing unit, and executes various programs, and controls the respective components. That is, the CPU 12 reads a program from the storage device 16, and executes the program, using the memory 14 as a work area. The CPU 12 performs the above-described various types of arithmetic processing in accordance with the programs stored in the storage device 16.

The memory 14 is formed with a random access memory (RAM), and, as a work area, temporarily stores a program and data. The storage device 16 is formed with a read only memory (ROM), and a hard disk drive (HDD), a solid state drive (SSD), or the like, and stores various programs including an operating system and various kinds of data.

The input/output I/F 18 is an interface that inputs data from an external device, and outputs data to an external device. Further, an input device for performing various inputs, such as a keyboard or a mouse, and an output device for outputting various kinds of information, such as a display or a printer, may be connected to the input/output I/F 18. A touch panel display may be adopted as an output device, and the input/output I/F 18 may be made to function as an input device.

The storage medium reading device 20 reads data stored in various kinds of storage media such as a compact disc (CD)-ROM, a digital versatile disc (DVD)-ROM, a Blu-ray disc, and a universal serial bus (USB) memory, and writes data into a storage medium, for example.

The communication I/F 22 is an interface for communicating with other devices, and a standard such as Ethernet (registered trademark), FDDI, or Wi-Fi (registered trademark) is used.

The information processing device 10 according to the present embodiment trains an autoencoder including an encoder and a decoder, using a technique as described above. As a result, it is possible to express a field representing the structure of a substance formed with an atomic point cloud, using a neural network model.

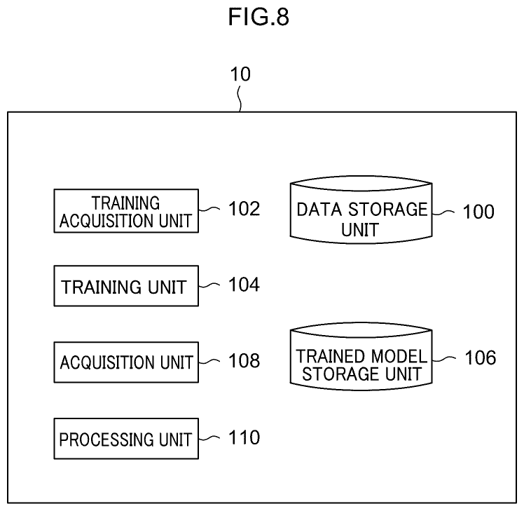

Next, functional components of the information processing device 10 are described. As illustrated in FIG. 8, the information processing device 10 functionally includes a training acquisition unit 102, a training unit 104, an acquisition unit 108, and a processing unit 110. Furthermore, a data storage unit 100 and a trained model storage unit 106 are provided in a predetermined storage area in the information processing device 10. The respective functional components are implemented by the CPU 12 reading the respective programs stored in the storage device 16, loading the programs into the memory 14, and executing the programs.

First, the information processing device 10 trains a neural network model expressing a field representing the structure of a substance formed with an atomic point cloud.

The data storage unit 100 stores training crystal data representing crystal structures of substances. The training crystal data includes position data indicating positions of atoms constituting crystals of substances, species data representing species of atoms constituting the crystals of the substances, and lattice constant data of the crystals of the substances.

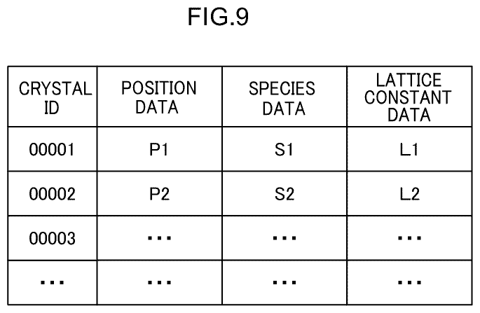

FIG. 9 is a table showing an example of a plurality of sets of training crystal data stored in the data storage unit 100. As shown in FIG. 9, one set of training crystal data is data representing a crystal structure of a substance. As shown in FIG. 9, in one set of training crystal data, position data indicating the positions of atoms constituting the crystal of a substance, species data representing the species of atoms constituting the crystal of the substance, and lattice constant data of the crystal of the substance are stored and are associated with one another. The position data is three-dimensional position coordinate data of the respective atoms in the plurality of atoms. The species data is label data indicating the species of the respective atoms in the plurality of atoms. The lattice constant data includes the length of the crystal axis and the inter-axial angle.

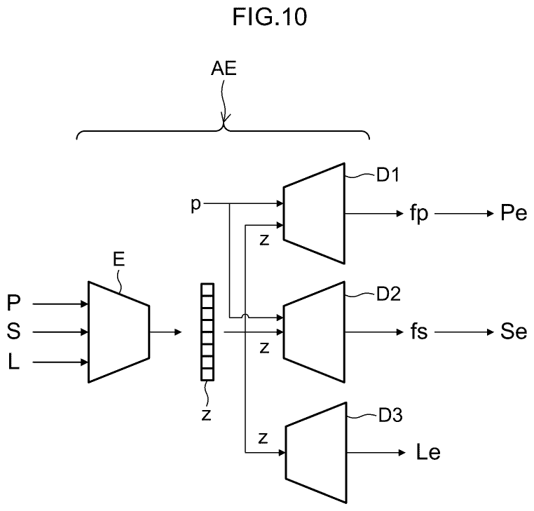

FIG. 10 is a diagram for explaining the structure of an autoencoder of the present embodiment, and an outline of a process to be performed by the information processing device 10 of the embodiment. FIG. 10 is a diagram in which FIG. 1(b) is further simplified.

As shown in FIG. 10, an autoencoder AE of the present embodiment includes an encoder E, a first decoder D1, a second decoder D2, and a third decoder D3.

As shown in FIG. 10, when a combination of position data P indicating the positions of the atoms constituting the crystal of a substance, species data S indicating the species of the atoms constituting the crystal of the substance, and lattice constant data L of the crystal of the substance is input, the encoder E outputs a latent vector z. One set of crystal data indicating the combination of the position data P, the species data S, and the lattice constant data L is set for the crystal structure of one substance.

Also, as shown in FIG. 10, when a combination of a query point p as the attention point in the substance and the latent vector z is input, the first decoder D1 outputs a position field fₚ indicating the field of the positions of the atoms constituting the crystal of the substance. On the basis of this position field fₚ, estimated position data Pₑ of the atoms constituting the crystal of the substance is computed. The computation method will be described later.

Further, as shown in FIG. 10, when a combination of the query point p as the attention point in the substance and the latent vector z is input, the second decoder D2 outputs a species field fₛ indicating the field of the species of the atoms constituting the crystal of the substance. On the basis of this species field fₛ, estimated species data Sₑ of the atoms constituting the crystal of the substance is computed. The computation method will be described later.

Also, as shown in FIG. 10, when the query point p as the attention point in the substance and the latent vector z are input, the third decoder D3 outputs estimated lattice constant data Lₑ. Although a case where a single third decoder D3 is adopted is described as an example in the present embodiment, there may be two third decoders D3. In this case, the third decoders D3 are a decoder that outputs the length of the crystal axis, and a decoder that outputs the inter-axial angle, for example.

The information processing device 10 of the present embodiment trains each parameter of the autoencoder AE by unsupervised machine learning (alternatively, self-supervised learning) so that the combination of the position data P, the species data S, and the lattice constant data L input to the autoencoder AE matches the combination of the estimated position data Pₑ, the estimated species data Sₑ, and the estimated lattice constant data Lₑ output from the autoencoder AE. As a result, a trained autoencoder AE is obtained. Also, a trained first decoder D1, a trained second decoder D2, and a trained third decoder D3, which are components of the trained autoencoder AE, are obtained.

Receiving an instruction signal to train the autoencoder AE, the training acquisition unit 102 reads the training crystal data stored in the data storage unit 100. The training acquisition unit 102 also sets a training query point that is an attention point in the learning substance. Note that the training acquisition unit 102 may set the training query point by randomly sampling positions in the space in a substance for training.

When training the autoencoder AE using unsupervised machine learning, the training unit 104 obtains the latent vector z representing the crystal structure of the substance for training, by inputting the training crystal data to the encoder E in the autoencoder AE. Note that the encoder E can be formed by using the idea of the above-described PointNet [28] or DeepSets [29], for example.

Successively, the training unit 104 inputs the combination of the latent vector z and the training query point p to the first decoder D1 of the autoencoder AE, and thus, obtains the position field fₚ representing the field of the positions of the atoms constituting the crystal of the substance for training. The training unit 104 then estimates the positions of the atoms constituting the crystal of the substance for training, on the basis of the position field fₚ output from the first decoder D1.

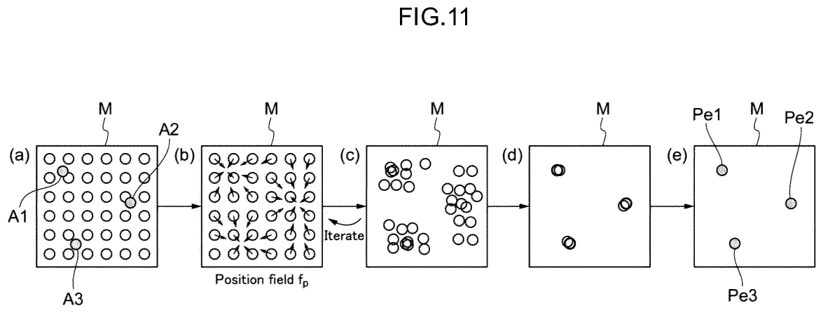

FIG. 11 is a diagram for explaining the setting of the training query point and the position field. Note that FIG. 11 is a diagram similar to one in FIG. 2, but is referred to again herein for ease of explanation. As illustrated in FIG. 11, a plurality of training query points is set in a space M in the substance. White circles shown in FIG. 11 represent the training query points. A1, A2, and A3 shown in FIG. 11(a) represent actual positions of atoms in the space M in the substance.

The training unit 104 obtains the position field fₚ as shown in FIG. 11(b) by inputting the combination of the training query point p and the latent vector z to the first decoder D1. Each of the arrows shown in FIG. 11 corresponds to the position field fₚ.

As illustrated in FIGS. 11(b) and 11(c), the training unit 104 then iterates updating of the position of each query point among the plurality of training query points, in accordance with the position field fₚ output from the first decoder D1. Specifically, the training unit 104 generates a new position of the training query point by adding a vector represented by the position field fₚ to the position of each query point among the plurality of training query points. As a result, the positions of the respective query points among the plurality of training query points converge to the positions of actual atoms, as illustrated in FIG. 11(d). On the basis of the respective positions of the training query points, the training unit 104 obtains estimated positions Pₑ1, Pₑ2, and Pₑ3 of atoms as shown in FIG. 11(e), using Non-max Suppression, for example.

Successively, the training unit 104 inputs the combination of the latent vector z and the training query point p to the second decoder D2 of the autoencoder AE, and thus, obtains the species field fₛ representing the field of the species of the atoms constituting the crystal of the substance for training. The training unit 104 then estimates the species of the atoms constituting the crystal of the substance for training, on the basis of the species field fₛ output from the second decoder D2.

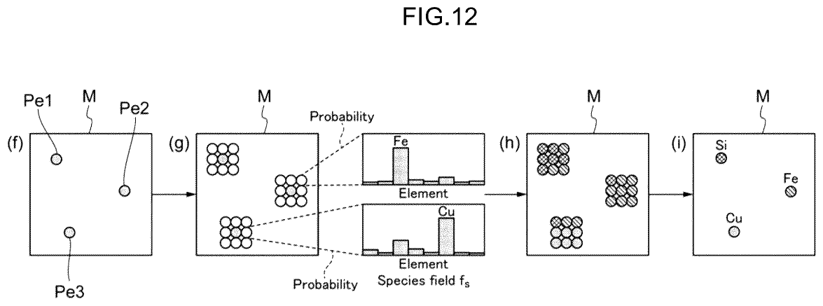

FIG. 12 is a diagram for explaining the setting of the training query point and the species field. Note that FIG. 12 is a diagram similar to one in FIG. 2, but is referred to again herein for ease of explanation. As illustrated in FIG. 12, a plurality of training query points is set in the space M in the substance. White circles shown in FIG. 12 represent the training query points, as in FIG. 12.

Specifically, as illustrated in FIGS. 12(f) and 12(g), the training unit 104 sets a plurality of training query points for the positions of estimated positions Pₑ1, Pₑ2, and Pₑ3 of atoms, and positions around the estimated positions Pₑ1, Pₑ2, and Pₑ3.

The training unit 104 then obtains the species field fₛ by inputting the combination of the training query point p and the latent vector z to the second decoder D2. As illustrated in FIG. 12(g), the species field fₛ corresponds to the probability of indicating the atomic species obtained at each training query point. The example illustrated in FIGS. 12(g) and 12(h) show that the probability that the species of the atom located at a training query point is iron Fe is the highest, and the probability that the species of the atom located at another training query point is copper Cu is the highest.

As illustrated in FIGS. 12(g) and 12(h), the training unit 104 then estimates the species of the atoms corresponding to the respective positions of the training query points, in accordance with the species field fₛ output from the second decoder D2. For example, for each of the estimated positions Pₑ1, Pₑ2, and Pₑ3, the training unit 104 identifies the element having the highest probability at the training query point corresponding to the estimated position, and the element having the highest probability at the training query points around the estimated position. The training unit 104 then estimates the most frequently appearing element at the plurality of training query points set for each of the estimated positions Pₑ1, Pₑ2, and Pₑ3 as the species of the atoms at the estimated positions Pₑ1, Pₑ2, and Pₑ3. As a result, an estimated species Sₑ1 of the atom present at the estimated position Pₑ1, an estimated species Sₑ2 of the atom present at the estimated position Pₑ2, and an estimated species Sₑ3 of the atom present at the estimated position Pₑ3 are obtained.

For example, as illustrated in FIG. 12(i), the species of the atom located at the estimated position Pₑ1 is estimated to be silicon Si, the species of the atom located at the estimated position Pₑ2 is estimated to be iron Fi, and the species of the atom located at the estimated position Pₑ3 is estimated to be copper Cu.

Successively, the training unit 104 obtains lattice constant data Lₑ of the crystal of the substance for training, by inputting the latent vector z to the third decoder D3 of the autoencoder AE. The lattice constant data Lₑ output from the third decoder D3 includes the length of the crystal axis and the inter-axial angle.

The training unit 104 then trains the autoencoder AE using unsupervised machine learning so that the combination of the estimated atomic positions, the estimated atomic species, and the lattice constant data output from the third decoder D3 corresponds to the combination of the atomic positions, the atomic species, and the lattice constant data in the training crystal data, and thus, produces the trained first decoder D1 and the trained second decoder D2.

For example, in the above example, the training unit 104 trains the autoencoder AE using unsupervised machine learning so that the estimated positions Pₑ1, Pₑ2, and Pₑ3 of atoms match the position data P1, P2, and P3 of atoms in the training crystal data, the estimated species Sₑ1, Sₑ2, and Sₑ3 of atoms match the species data S1, S2, and S3 of atoms in the training crystal data, and the lattice constant data Lₑ output from the third decoder D3 matches the lattice constant data L in the training crystal data, and thus, produces the trained first decoder D1 and the trained second decoder D2.

The training unit 104 stores the trained autoencoder AE into the trained model storage unit 106. Because the trained autoencoder AE also includes the trained first decoder D1, the trained second decoder D2, and the trained third decoder D3, these trained models are also stored into the trained model storage unit 106.

Note that the trained encoder E outputs the latent vector z representing the crystal of the substance when crystal data is input, the crystal data being crystal data representing the crystal structure of the substance and including position data indicating the positions of the atoms constituting the crystal of the substance, species data indicating the species of the atoms constituting the crystal of the substance, and lattice constant data of the crystal of the substance.

As described above, the field data representing the field of the structure of the substance of the present embodiment is indicated by the position field representing the field of the positions of the atoms constituting the crystal of the substance and the species field representing the field of the species of the atoms constituting the crystal of the substance. Therefore, when an arbitrary vector and a query point are input, the trained first decoder D1, which is an example of the trained first neural network model, outputs the position field fₚ corresponding to the query point. Also, when an arbitrary vector and a query point are input, the second decoder D2, which is an example of the trained second neural network model, outputs the species field fₛ corresponding to the query point. Note that the trained first decoder D1 and the trained second decoder D2 are an example of the trained neural network model that outputs field data representing the field of the structure of the substance at a query point when the arbitrary vector and the query point are input.

When the combination of an arbitrary vector in place of the latent vector z and a query point is input, the trained third decoder outputs the lattice constant data Lₑ of the crystal of the substance.

Receiving an instruction signal to obtain the field data of the crystal structure of the target substance, the acquisition unit 108 reads the trained first decoder D1 and the trained second decoder D2 stored in the trained model storage unit 106. The acquisition unit 108 also acquires the target vector input from the user and a query point which is an attention point in the substance.

Note that the target vector is an arbitrary vector in place of the latent vector z, and may be any vector that describes the properties of the material in the same manner as a latent vector does. For example, the target vector may be a vector representing physical properties desired by the user. For example, [bandgap=xxx] may be stored in a first component of the target vector, and [formation energy=xxx] may be stored in a second component. In this case, the trained first decoder D1 and the trained second decoder D2 are treated as preliminary trained models, and are retrained by a vector representing the physical properties of substances. Thus, useful field data is output from the trained first decoder D1 and the trained second decoder D2.

The processing unit 110 obtains the field data corresponding to the query point by inputting the target vector and the query point acquired by the acquisition unit 108 to the trained first decoder D1 and the trained second decoder D2.

Specifically, the processing unit 110 obtains the position field fₚ corresponding to the query point by inputting the target vector and the query point acquired by the acquisition unit 108 to the trained first decoder D1. The processing unit 110 also obtains the species field fₛ corresponding to the query point by inputting the target vector and the query point acquired by the acquisition unit 108 to the trained second decoder D2.

The field data output from the trained first decoder D1 and the trained second decoder D2 is data that represents the field of the structure of the substance in accordance with the target vector and the query point. By inputting the target vector that is the arbitrary vector in place of the latent vector to the trained first decoder D1 and the trained second decoder D2, it is possible to predict the crystal structure represented by the target vector, for example.

In a case where the target vector is a vector representing desired physical properties as described above, for example, field data corresponding to the vector is output. Because a plurality of query points is input to the trained second decoder D2 and the trained second decoder D2 to obtain the field data at the plurality of query points, it is also possible to produce a crystal structure of a substance having the desired physical properties on the basis of the field data.

As the target vector, measurement data of X-ray diffraction (XRD) can also be used, for example. In this case, the XRD measurement data of the target substance is input to the trained first decoder D1 and the trained second decoder D2. Thus, the crystal structure of the target substance can be predicted. Furthermore, the trained first decoder D1 and the trained second decoder D2 of the present embodiment can also be used in combination with an encoder that processes some other modal such as text data.

### <Operations of Information Processing Device 10>

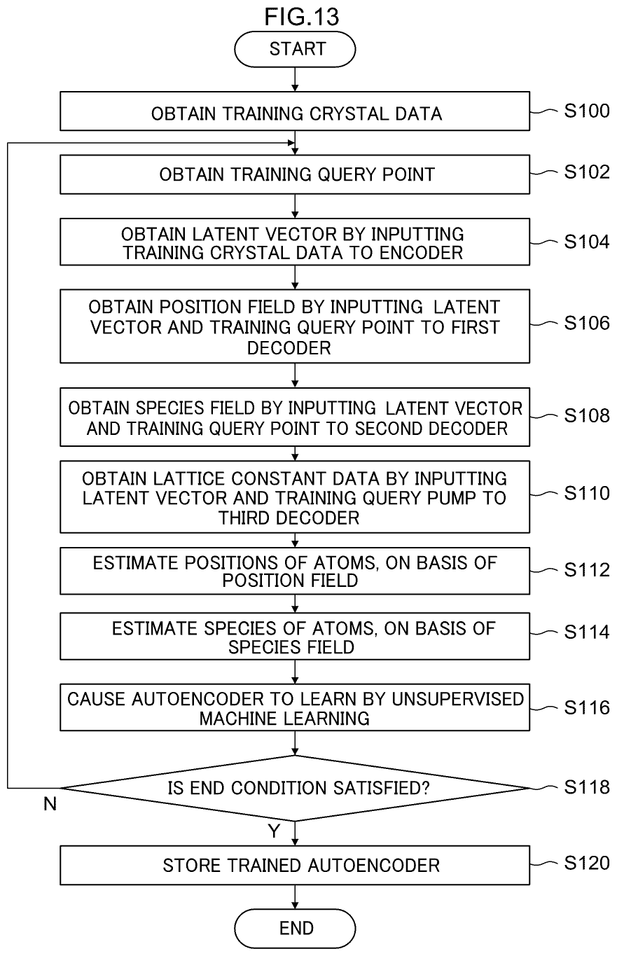

Next, operations of the information processing device 10 according to the embodiment are described with reference to the drawings. Receiving the training crystal data, the information processing device 10 stores the training crystal data into the data storage unit 100. The information processing device 10 then receives an instruction signal to start a training process, and executes a trained model generation process routine shown in FIG. 13.

### <Trained Model Generation Process Routine>

In step S100, the training acquisition unit 102 acquires a plurality of sets of the training crystal data stored in the data storage unit 100.

In step S102, the training acquisition unit 102 sets one set of the training crystal data from among the plurality of sets of the training crystal data acquired in step S100. The training unit 104 then sets a plurality of training query points pᵢ in the space in the substance indicated by the set training crystal data. Note that i is an index for identifying the training query points.

In step S104, the training unit 104 obtains the latent vector z by inputting the position data P, the species data S, and the lattice constant data L in the training crystal data set in step S102 to the encoder E of the autoencoder AE.

In step S106, the training unit 104 obtains the position field fₚ by inputting the combination of the training query points pᵢ set in step S102 and the latent vector z set in step S104 to the first decoder D1. Note that the training unit 104 obtains the position field fₚ for each query point among the plurality of the training query points.

In step S108, the training unit 104 obtains the species field fₛ by inputting the combination of the training query points pᵢ set in step S102 and the latent vector z set in step S104 to the second decoder D2. Note that the training unit 104 obtains the species field fₛ for each query point among the plurality of the training query points.

In step S110, the training unit 104 obtains the lattice constant data L_{c} by inputting the combination of the training query points pᵢ set in step S102 and the latent vector z set in step S104 to the third decoder D3. Note that the training unit 104 obtains the lattice constant data L_{c} for each query point among the plurality of the training query points.

In step S112, the training unit 104 estimates the positions of atoms on the basis of the position field fₚ obtained in step S106. Note that the training unit 104 updates the positions of the plurality of the training query points by the above-described method, and thus, obtains the final estimated positions Pₑ.

In step S114, the training unit 104 estimates the species of atoms, on the basis of the estimated positions Pₑ of atoms obtained in step S112 and the species field fₛ obtained in step S108. As a result, the training unit 104 obtains the estimated species Sₑ of the atoms present at the estimated positions Pₑ.

In step S116, the training unit 104 trains the autoencoder AE using unsupervised machine learning so that the estimated positions Pₑ of atoms obtained in step S112 match the position data P of atoms in the training crystal data, the estimated species Sₑ of atoms obtained in step S114 match the species data S of atoms in the training crystal data, and the lattice constant data L_{c} obtained in step S110 matches the lattice constant data L in the training crystal data, and thus, produces the trained first decoder D1 and the trained second decoder D2.

The process in steps S102 to S116 is repeated until a condition for an end of machine learning are satisfied. As the condition for an end of machine learning, it is possible to adopt a condition such as whether a machine learning process has been repeated a predetermined number of times, or whether the error between the data output from the autoencoder AE and the training crystal data is equal to or smaller than a predetermined threshold, for example. Although a case where one set of the training crystal data is set and training is then performed has been described above as an example, the embodiment is not limited to this, and machine learning may be performed using a plurality of sets of the training crystal data at once.

In step S118, the training unit 104 determines whether the above-described end condition is satisfied. If the end condition is satisfied, the operation proceeds to step S120. If the end condition is not satisfied, on the other hand, the operation returns to step S102.

In step S120, the training unit 104 stores the trained autoencoder AE obtained by the machine learning process in steps S102 to S116, into the trained model storage unit 106.

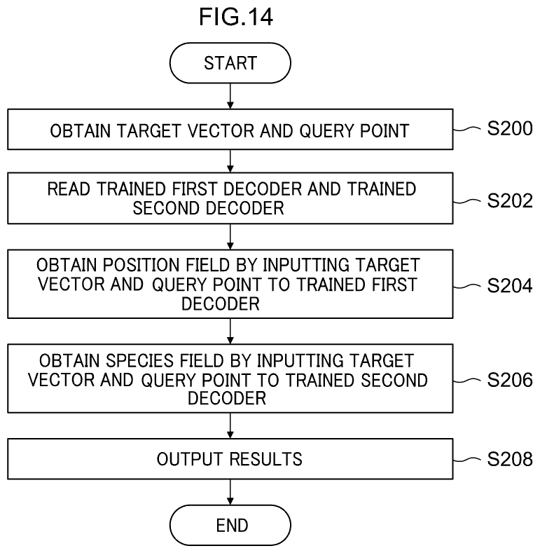

Next, receiving the target vector and a query point, the information processing device 10 performs an estimation process routine shown in FIG. 14.

### <Estimation Process Routine>

In step S200, the acquisition unit 108 acquires the target vector and a query point.

In step S202, the processing unit 110 reads the trained first decoder D1 and the trained second decoder D2 stored in the trained model storage unit 106.

In step S204, the processing unit 110 obtains the position field fₚ corresponding to the target vector and the query point, by inputting the target vector and the query point acquired in step S200 to the trained first decoder D1 read in step S202.

In step S206, the processing unit 110 obtains the species field fₛ corresponding to the target vector and the query point, by inputting the target vector and the query point acquired in step S200 to the trained second decoder D2 read in step S202.

In step S208, the processing unit 110 outputs the position field fₚ acquired in step S204 and the species field fₛ acquired in step S206 as the results.

As described above, with the information processing device 10, it is possible to train a neural network model expressing a field representing the structure of a substance formed with an atomic point cloud. Also, with the information processing device 10, it is possible to express a field representing the structure of a substance formed with an atomic point cloud, using a neural network model.

Specifically, the information processing device 10 obtains training crystal data that is training crystal data representing the crystal structure of a substance for training and includes position data representing the positions of atoms constituting the crystal of the substance for training, species data representing the species of the atoms constituting the crystal of the substance for training, and lattice constant data of the crystal of the substance for training, and a training query point that is an attention point in the substance for training. When training an autoencoder using unsupervised machine learning, the information processing device 10 obtains a latent vector representing the crystal structure of the substance for training, by inputting the training crystal data to the encoder of the autoencoder. The information processing device 10 inputs the combination of the latent vector and the training query point to the first decoder of the autoencoder, and thus, obtains a position field representing the field of the positions of the atoms constituting the crystal of the substance for training. The information processing device 10 estimates the positions of the atoms constituting the crystal of the substance for training, on the basis of the position field output from the first decoder. The information processing device 10 inputs the combination of the latent vector and the training query point to the second decoder of the autoencoder, and thus, obtains species field data representing the field of the species of the atoms constituting the crystal of the substance for training. The information processing device 10 estimates the species of the atoms constituting the crystal of the substance for training, on the basis of the species field output from the second decoder. The information processing device 10 inputs the combination of the latent vector and the training query point to the third decoder of the autoencoder, and thus, obtains lattice constant data of the crystal of the substance for training. The information processing device 10 trains the autoencoder using unsupervised machine learning so that the combination of the estimated atomic positions, the estimated atomic species, and the lattice constant data output from the third decoder corresponds to the combination of the position data, the species data, and the lattice constant data in the training crystal data, and thus, produces the trained first decoder and the trained second decoder.

In the present embodiment, by adopting the method as described above, a field representing the structure of a substance can be expressed with a neural field. Also, it is possible to generate crystal data from a fixed-length vector.

Also, the information processing device 10 obtains a target vector and a query point that is an attention point in a substance, and acquires field data corresponding to the query point by inputting the target vector and the query point to the first decoder and the second decoder that are an example of the trained neural network model. This field data is indicated by the position field representing the field of the positions of atoms constituting the crystal of the substance and the species field representing the field of the species of atoms constituting the crystal of the substance. When the arbitrary vector in place of the latent vector and the query point are input, the first decoder outputs a position field corresponding to the query point. When the arbitrary vector in place of the latent vector and the query point are input, the second decoder outputs a species field corresponding to the query point. The information processing device 10 obtains the position field corresponding to the query point by inputting the target vector and the query point to the first decoder. The information processing device 10 also obtains the species field corresponding to the query point by inputting the target vector and the query point to the second decoder. In a case where the target vector is a vector representing desired physical properties, for example, field data corresponding to the vector is output. Because a plurality of query points is input to the trained second decoder D2 and the trained second decoder D2 to obtain the field data at the plurality of query points, it is also possible to produce a crystal structure of a substance having the desired physical properties on the basis of the field data.

In a case where the target vector is changed, it is preferable to retrain the trained first decoder D1 and the trained second decoder D2. In this case, the trained first decoder D1 and the trained second decoder D2 are used as preliminary trained models.

Although a case where a field representing a crystal structure is modeled with a neural field has been described as an example in the above embodiment, the embodiment is not limited to this. A crystal structure including iterative structures is not necessarily used, but the structure of a substance formed with an atomic point cloud not including iterative structures may be modeled with a neural field. In this case, estimation of the lattice constant may be unnecessary, and thus, the lattice decorder shown in FIG. 1 may be unnecessary.

Although a case where the vector z input to the decoders is a latent vector output from the encoder has been described as an example in the above embodiment, the embodiment is not limited to this. For example, a vector representing physical properties desired by the user may be z, and the vector z may be given to the decoders (for example, conditions such as bandgap=xxx and formation energy=xxx are expressed by a vector). Alternatively, random noise may be used as the vector z.

Although a case where an autoencoder including an encoder and decoders is used as a neural network model has been described as an example in the above embodiment, the embodiment is not limited to this. As the neural network model, some other model may be used, and, for example, a generative adversarial network (GAN) may be used.

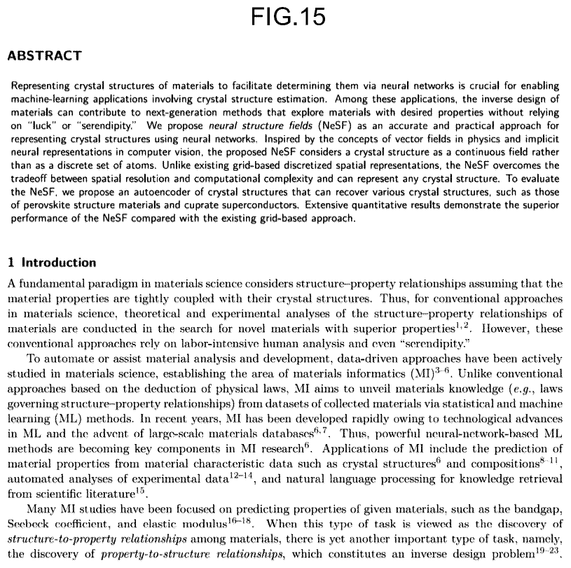

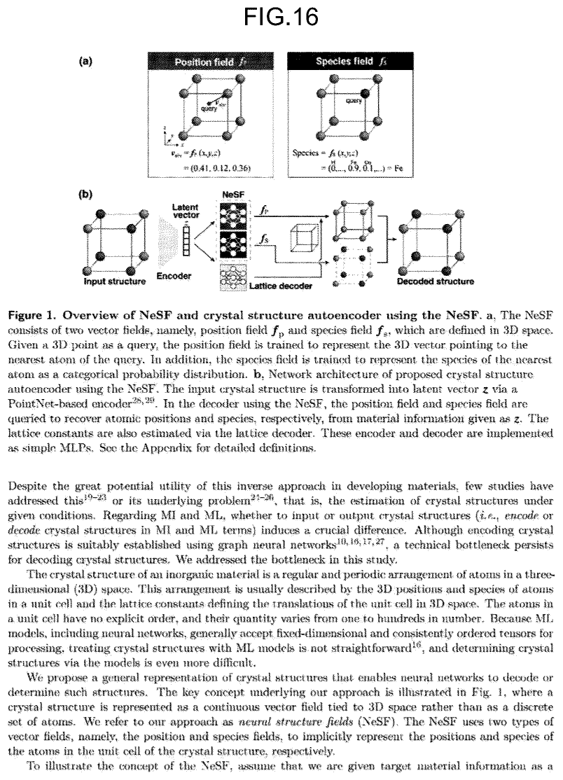

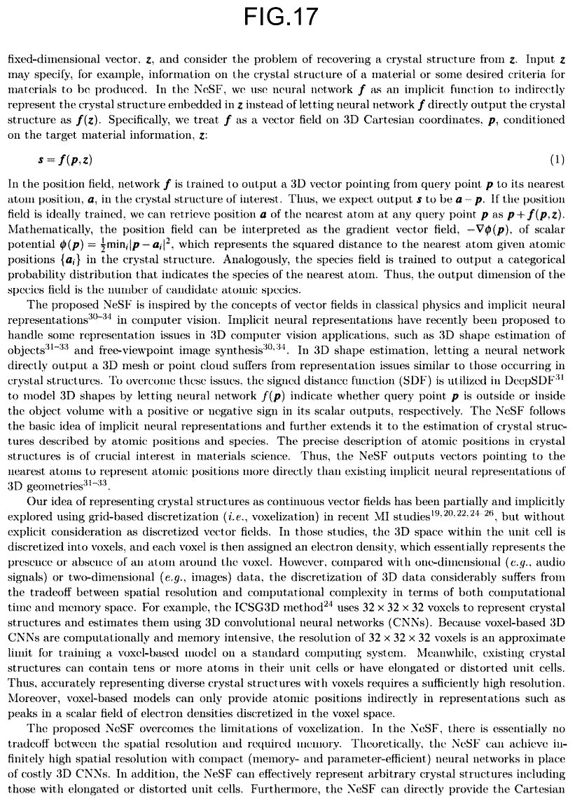

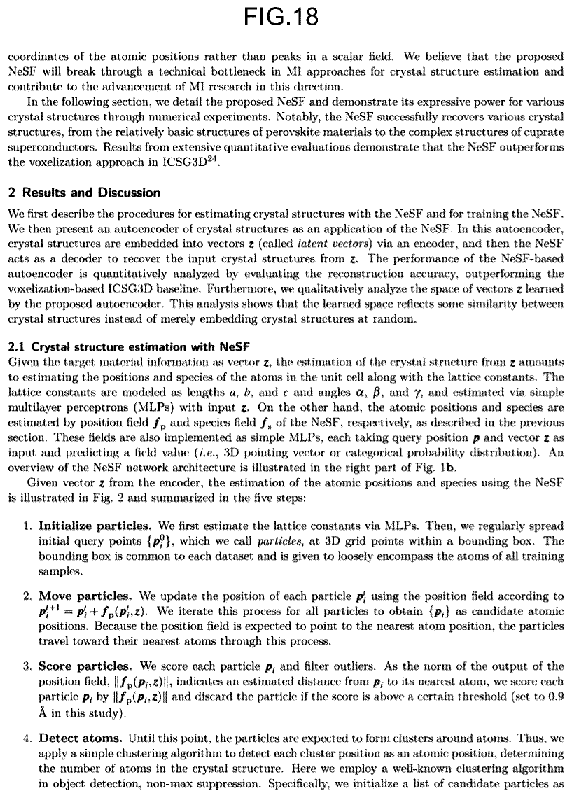

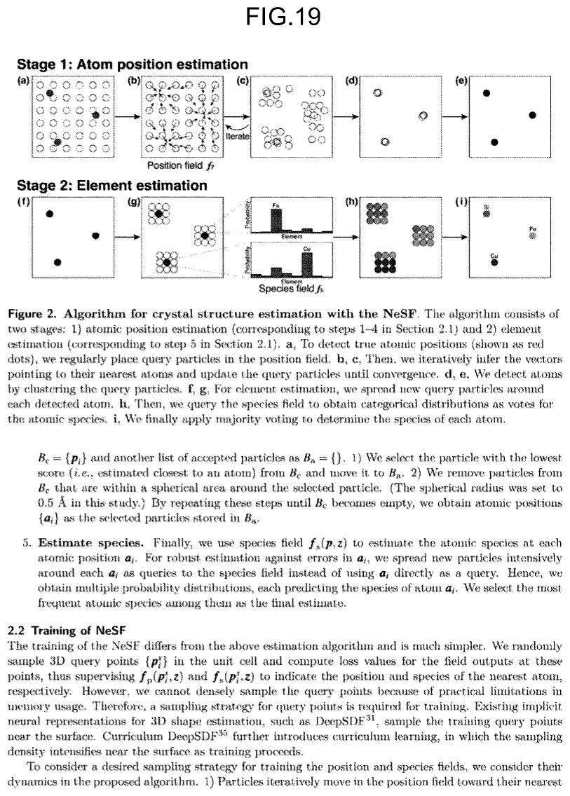

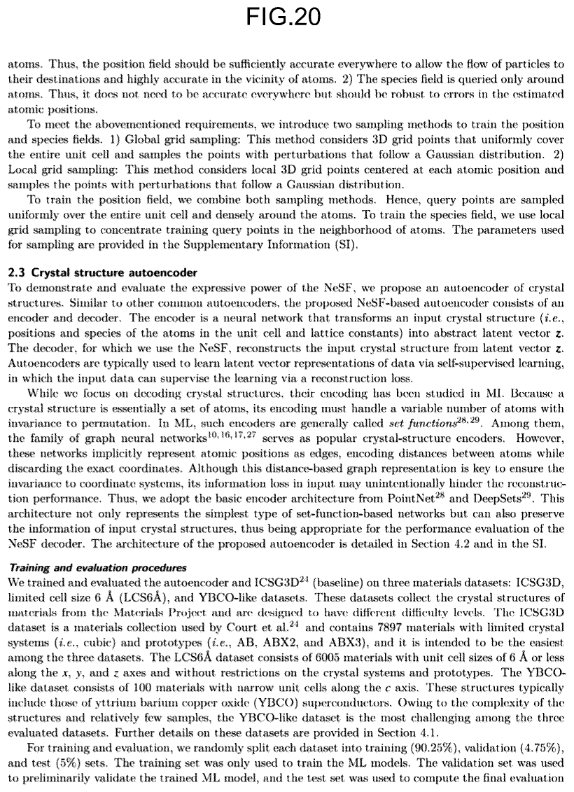

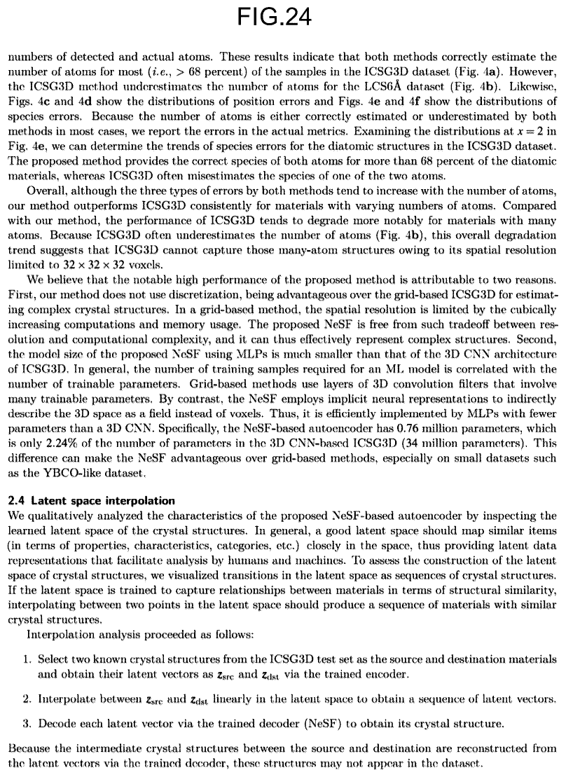

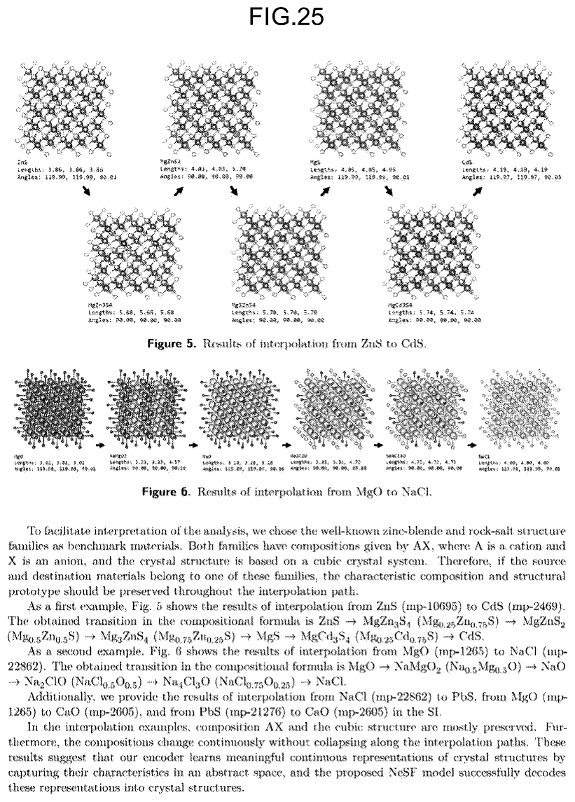

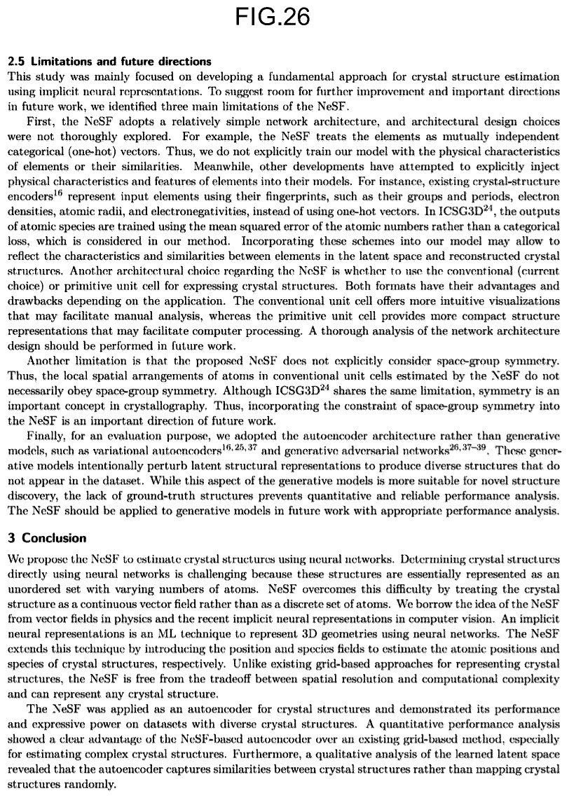

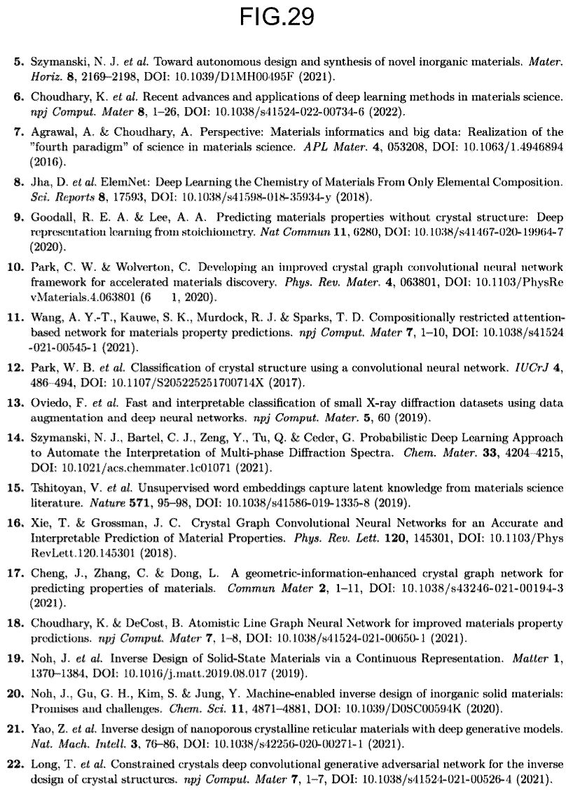

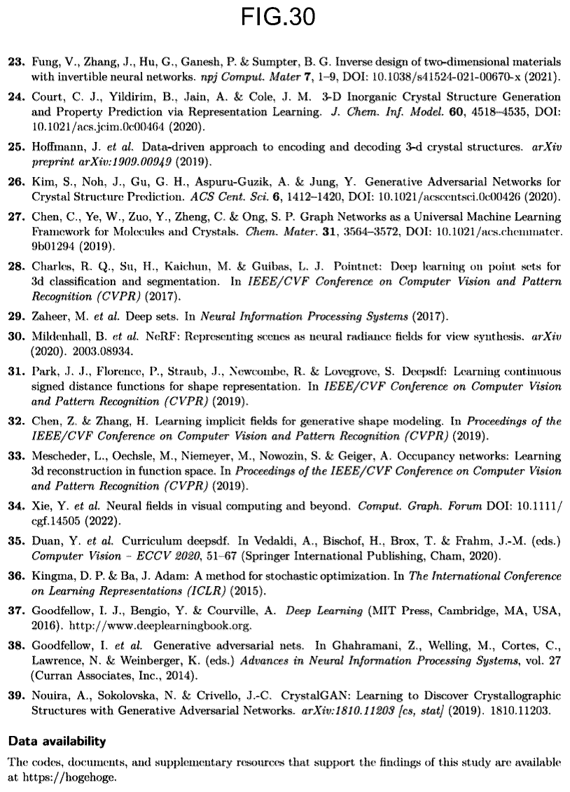

FIGS. 15 to 30 are charts for explaining the present embodiment in detail.

Alternatively, the respective processes performed by the CPU reading software (programs) in the above embodiment may be performed by various processors other than a CPU. Examples of the processors in this case include a programmable logic device (PLD) in which the circuit configuration can be changed after manufacturing, such as a field-programmable gate array (FPGA), and a dedicated electric circuit that is a processor having a circuit configuration designed exclusively for performing specific processing, such as an application specific integrated circuit (ASIC). Further, each process may be performed by one of these various processors, or by a combination of two or more processors of the same type or different types (for example, a plurality of FPGAs, or a combination of a CPU and an FPGA). More specifically, the hardware structure of any of these various processors is an electric circuit in which circuit elements such as semiconductor elements are combined.

In the above embodiment, a mode in which each program is stored (installed, for example) beforehand into a storage device has been described, but the embodiment is not limited to this. The programs may be provided in a form stored in a storage medium such as a CD-ROM, a DVD-ROM, a Blu-ray Disc, or a USB memory. Alternatively, the programs may be downloaded from an external device via a network.

### Supplementary Notes

In the following, modes of the present disclosure are noted.

### (Supplementary Note 1)

An information processing device including a processing unit that expresses a field representing a structure of a substance formed with an atomic point cloud, using a neural network model.

### (Supplementary Note 2)

The information processing device of supplementary note 1, further including
an acquisition unit that acquires a target vector and a query point that is an attention point in the substance, in which
the neural network model is a trained neural network model that outputs field data representing a field of the structure of the substance at the query point when an arbitrary vector and the query point are input, and
the processing unit obtains the field data corresponding to the query point by inputting the target vector and the query point acquired by the acquisition unit to the trained neural network model.

### (Supplementary Note 3)

The information processing device of supplementary note 2, in which
the field data is represented by a position field representing a field of positions of atoms constituting crystal of the substance and a species field representing a field of species of atoms constituting the crystal of the substance,
the trained neural network model includes a trained first neural network model and a trained second neural network model,
the trained first neural network model outputs the position field corresponding to the query point when the arbitrary vector and the query point are input,
the trained second neural network model outputs the species field corresponding to the query point when the arbitrary vector and the query point are input, and
the processing unit
obtains the position field corresponding to the query point by inputting the target vector and the query point acquired by the acquisition unit to the trained first neural network model, and
obtains the species field corresponding to the query point by inputting the target vector and the query point acquired by the acquisition unit to the trained second neural network model.

### (Supplementary Note 4)

The information processing device of supplementary note 2 or 3, in which
the trained neural network model
is a trained model produced beforehand by machine learning on the basis of training crystal data that is crystal data representing a crystal structure of the substance and includes position data representing positions of atoms constituting crystal of the substance, species data representing species of atoms constituting the crystal of the substance, and lattice constant data of the crystal of the substance.

### (Supplementary Note 5)

The information processing device of supplementary note 3, in which
the trained neural network model is a trained first decoder and a trained second decoder of a trained autoencoder,
a trained encoder of the trained autoencoder outputs a latent vector representing the crystal of the substance when crystal data representing a crystal structure of the substance is input, the crystal data including position data representing positions of atoms constituting the crystal of the substance, species data representing species of atoms constituting the crystal of the substance, and lattice constant data of the crystal of the substance,
the trained first decoder outputs the position field corresponding to the query point when the arbitrary vector in place of the latent vector and the query point are input,
the trained second decoder outputs the species field corresponding to the query point when the arbitrary vector in place of the latent vector and the query point are input, and
the trained first decoder and the trained second decoder are trained models obtained by training an autoencoder by unsupervised machine learning on the basis of the crystal data, the autoencoder including an encoder, a first decoder, and a second decoder.

### (Supplementary Note 5)

The information processing device of supplementary note 4, in which
the autoencoder further includes a third decoder, and
a trained third decoder is a trained model that outputs lattice constant data of the crystal of the substance when the arbitrary vector in place of the latent vector is input.

### (Supplementary Note 7)

A trained model generation device including a training unit that trains a neural network model expressing a field representing a structure of a substance formed with an atomic point cloud.

### (Supplementary Note 8)

The trained model generation device of supplementary note 7, further including
a training acquisition unit that acquires training crystal data representing a crystal structure of the substance for training and a training query point that is an attention point in the substance for training, the training crystal data including position data representing positions of atoms constituting crystal of the substance for training, species data representing species of atoms constituting the crystal of the substance for training, and lattice constant data of the crystal of the substance for training, and,
when training an autoencoder using unsupervised machine learning,
the training unit
obtains a latent vector representing the crystal structure of the substance for training, by inputting the training crystal data to an encoder of the autoencoder,
obtains a position field representing a field of the positions of atoms constituting the crystal of the substance for training, by inputting a combination of the latent vector and the training query point to a first decoder of the autoencoder,
estimates the positions of atoms constituting the crystal of the substance for training, on the basis of the position field output from the first decoder,
obtains species field data representing a field of species of atoms constituting the crystal of the substance for training, by inputting the combination of the latent vector and the training query point to a second decoder of the autoencoder,
estimates species of atoms constituting the crystal of the substance for training, on the basis of the species field output from the second decoder,
obtains lattice constant data of the crystal of the substance for training, by inputting the combination of the latent vector and the training query point to a third decoder of the autoencoder,
and produces a trained first decoder and a trained second decoder by training the autoencoder using unsupervised machine learning so that a combination of the estimated positions of atoms, the estimated species of atoms, and the lattice constant data output from the third decoder corresponds to a combination of the position data, the species data, and the lattice constant data in the training crystal data.

### (Supplementary Note 9)

An information processing method implemented by a computer to perform a process including expressing a field representing a structure of a substance formed with an atomic point cloud, using a neural network model.

### (Supplementary Note 10)

A trained model generation method implemented by a computer to perform a process including training a neural network model expressing a field representing a structure of a substance formed with an atomic point cloud.

### (Supplementary Note 11)

An information processing program for causing a computer to perform a process including expressing a field representing a structure of a substance formed with an atomic point cloud, using a neural network model.

### (Supplementary Note 12)

A trained model generation program for causing a computer to perform a process including training a neural network model expressing a field representing a structure of a substance formed with an atomic point cloud.

The disclosures of Japanese Patent Application No. 2022-186043, filed on Nov. 21, 2022, and Japanese Patent Application No. 2023-130520, filed on Aug. 9, 2023, are incorporated herein by reference in their entirety. All literatures, patent applications, and technical standards mentioned in this specification are incorporated herein by reference to the same extent as that in a case where each literature, each patent application, and each technical standard are specifically and individually mentioned to be incorporated by reference.

1. An information processing device comprising a processing unit that expresses a field representing a structure of a substance formed with an atomic point cloud, using a neural network model.

2. The information processing device according to claim 1, further comprising
an acquisition unit that acquires a target vector and a query point that is an attention point in the substance, wherein
the neural network model is a trained neural network model that outputs field data representing a field of the structure of the substance at the query point when an arbitrary vector and the query point are input, and
the processing unit obtains the field data corresponding to the query point by inputting the target vector and the query point acquired by the acquisition unit to the trained neural network model.

3. The information processing device according to claim 2, wherein
the field data is represented by a position field representing a field of a position of an atom forming crystal of the substance and a species field representing a field of a species of the atom forming the crystal of the substance,
the trained neural network model includes a trained first neural network model and a trained second neural network model,
the trained first neural network model outputs the position field corresponding to the query point when the arbitrary vector and the query point are input,
the trained second neural network model outputs the species field corresponding to the query point when the arbitrary vector and the query point are input, and
the processing unit
obtains the position field corresponding to the query point by inputting the target vector and the query point acquired by the acquisition unit to the trained first neural network model, and
obtains the species field corresponding to the query point by inputting the target vector and the query point acquired by the acquisition unit to the trained second neural network model.

4. The information processing device according to claim 2, wherein
the trained neural network model
is a trained model produced beforehand by machine learning on a basis of training crystal data that is crystal data representing a crystal structure of the substance and includes position data representing a position of an atom forming crystal of the substance, species data representing a species of the atom forming the crystal of the substance, and lattice constant data of the crystal of the substance.

5. The information processing device according to claim 3, wherein
the trained neural network model is a trained first decoder and a trained second decoder of a trained autoencoder,
a trained encoder of the trained autoencoder outputs a latent vector representing the crystal of the substance when crystal data representing a crystal structure of the substance is input, the crystal data including position data representing a position of an atom forming the crystal of the substance, species data representing a species of the atom forming the crystal of the substance, and lattice constant data of the crystal of the substance,
the trained first decoder outputs the position field corresponding to the query point when the arbitrary vector in place of the latent vector and the query point are input,
the trained second decoder outputs the species field corresponding to the query point when the arbitrary vector in place of the latent vector and the query point are input, and
the trained first decoder and the trained second decoder are trained models obtained by training an autoencoder by unsupervised machine learning on a basis of the crystal data, the autoencoder including an encoder, a first decoder, and a second decoder.

6. The information processing device according to claim 5, wherein
the autoencoder further includes a third decoder, and
a trained third decoder is a trained model that outputs lattice constant data of the crystal of the substance when the arbitrary vector in place of the latent vector is input.

7. A trained model generation device comprising a training unit that trains a neural network model expressing a field representing a structure of a substance formed with an atomic point cloud.

8. The trained model generation device according to claim 7, further comprising
a training acquisition unit that acquires training crystal data representing a crystal structure of the substance for training and a training query point that is an attention point in the substance for training, the training crystal data including position data representing a position of an atom forming crystal of the substance for training, species data representing a species of the atom forming the crystal of the substance for training, and lattice constant data of the crystal of the substance for training, and,
when training an autoencoder using unsupervised machine learning,
the training unit
obtains a latent vector representing the crystal structure of the substance for training, by inputting the training crystal data to an encoder of the autoencoder,
obtains a position field representing a field of a position of an atom forming the crystal of the substance for training, by inputting a combination of the latent vector and the training query point to a first decoder of the autoencoder,
estimates the position of the atom forming the crystal of the substance for training, on a basis of the position field output from the first decoder,
obtains species field representing a field of a species of the atom forming the crystal of the substance for training, by inputting the combination of the latent vector and the training query point to a second decoder of the autoencoder,
estimates the species of the atom forming the crystal of the substance for training, on a basis of the species field output from the second decoder,
obtains lattice constant data of the crystal of the substance for training, by inputting the combination of the latent vector and the training query point to a third decoder of the autoencoder,
and produces a trained first decoder and a trained second decoder by training the autoencoder using unsupervised machine learning so that a combination of the estimated position of the atom, the estimated species of the atom, and the lattice constant data output from the third decoder corresponds to a combination of the position data, the species data, and the lattice constant data in the training crystal data.

9. An information processing method implemented by a computer to perform a process including expressing a field representing a structure of a substance formed with an atomic point cloud, using a neural network model.

10. A trained model generation method implemented by a computer to perform a process including training a neural network model expressing a field representing a structure of a substance formed with an atomic point cloud.

11. A non-transitory storage medium storing an information processing program that is executable by a computer to perform a process including expressing a field representing a structure of a substance formed with an atomic point cloud, using a neural network model.

12. A non-transitory storage medium storing a trained model generation program that is executable by a computer to perform a process including training a neural network model expressing a field representing a structure of a substance formed with an atomic point cloud.
